(19) United States
(12) Patent Application Publication
(10) Pub. No.: US 2022/0315634 A1
(43) Pub. Date: Oct. 6, 2022

(54) TGF-BETA VACCINE

(71) Applicant: IO BIOTECH APS, Copenhagen (DK)

(72) Inventor: Mads Hald ANDERSEN, Naerum (DK)

(21) Appl. No.: 17/616,382

(22) PCT Filed: Jun. 4, 2020
(86) PCT No.: PCT/EP2020/065472

§ 371 Date: Dec. 3, 2021

(30) Foreign Application Priority Data
Jun. 5, 2019 (GB) 1908012.6

(51) Int. Cl. C07K 14/495; A61K 39/00; A61P 37/04; A61P 35/00

(52) U.S. Cl. CPC C07K 14/495; A61K 39/001134; A61P 37/04; A61P 35/00; A61K 2039/572

(57) ABSTRACT

The present invention relates to novel polypeptides derived from TGFb1. The invention also concerns uses of the polypeptides, polynucleotides encoding the peptides and uses thereof, and compositions comprising the polypeptides and uses thereof.

### FIELD OF THE INVENTION

The present invention relates to novel polypeptides, which are derived from transforming growth factor beta 1 (TGFβ1; TGFb1) as well as polynucleotides encoding such polypeptides and compositions comprising such peptides. The invention also concerns uses, and methods of using, said polypeptides, polynucleotides, and compositions.

### BACKGROUND OF THE INVENTION

TGFb is a multifunctional cytokine with a key role in the regulation of the immune system. There are four isoforms, of which isoform1 (TGFb1) is particularly important in T-cell immunity. In the context of cancer, TGFb1 disarms various immune cells like cytotoxic T-cells (CTLs), tumor-associated neutrophils and Natural Killer (NK) cells. It also contributes to tumor vascularization and metastasis. Consequently, TGFb1 is a key inhibitory molecule in the tumor microenvironment (TME), contributing to a down-regulation of the immune system's anti-tumor machinery and enabling immune-evasion by cancer cells.

In recent studies of murine models of metastatic liver cancer, TGFb1 has also been seen to contribute to a decrease in the efficiency as cancer therapies of Immune Checkpoint Blockers (ICBs), such as PD-L1 blockade.

### SUMMARY OF THE INVENTION

The polypeptides of the present invention are expected to be particularly effective at stimulating a beneficial immune response against TGFb1-expressing cells. The development of novel immune therapies for cancer requires a thorough understanding of the molecules that are involved in the pathogenesis as well as the specific proteins recognized by the immune system. In the clinical setting the induction of TGFb1-specific immune responses may directly kill TGFb1-expressing cancer cells, but more significantly it will support anti-cancer immune responses in general by suppressing the immune suppressive function of TGFb 1. Targeting TGFb1 and TGFb1-expressing cells, e.g. by vaccination with the polypeptides of the present invention, will consequently be highly synergistic with additional anti-cancer immunotherapy, such as Immune Checkpoint Blockers (ICBs).

TGFb1 is a dimeric cytokine which shares a cysteine knot structure connected together by intramolecular disulfide bonds. TGFb1 is synthesized as a monomeric 390-amino acid precursor protein, which is referred to interchangeably as: TGFb1 pre-protein; TGFb1 precursor; full-length TGFb1; pre-pro-TGFb1. The full-length sequence of the TGFb1 pre-protein is provided as SEQ ID NO: 1.

The TGFb1 pre-protein monomer has a molecular weight of about 25 kDa. The TGFb1 protein monomer has three distinct domains: the signal peptide (SP: amino acids 1-29; SEQ ID NO: 2), the latency associated peptide (LAP: amino acids 30-278; SEQ ID NO: 3) and the mature peptide (mature TGFb1: amino acids 279-390; SEQ ID NO: 4), as shown in FIG. 1E.

The TGFb1 SP targets the protein to a secretory pathway; the SP is cleaved off in the rough endoplasmatic reticulum. TGFb1 monomers comprising the LAP and mature TGFb1 may dimerize in the endoplasmic reticulum via disulfide bridges between cysteine residues in the LAP (e.g. Cys 223 and Cys 225) and the mature TGFb1 peptide (e.g. Cys 356) to form a TGFb1 homodimer. This TGFb1 homodimer is referred to as the small latent complex (SLC). The SLC may be bound by so-called latent TGF-β-Binding Protein (LTBP) to form a larger complex referred to as the large latent complex (LLC). The LLC may be secreted into extracellular media (ECM). However, the presence of LAP and the LTBP prevent TGFb1 from binding to, and activating, its extracellular receptors. Active TGFb1 consists of a homodimer of mature TGFb1 peptides. There are various mechanisms by which the mature TGFb1 homodimer is released from LAP and LTBP, which include degradation of LAP by proteases, induction of conformational change in LAP by interaction with thrombospondin, and rupture of noncovalent bonds between LAP and TGFβ-1.

An object of the present invention is the development of a T-cell-mediated mechanism for depriving TGFb1 from TME. The present inventors investigated the existence of spontaneous TGFb1-specific T-cell responses in vivo by screening PBMCs from healthy donors and cancer patients. TGFb1-specific T-cell populations were then be isolated, expanded and characterized by various assays regarding HLA restriction, cytokines production and cytotoxicity.

The present inventors have identified regions of human TGFb1 which have greatest immunogenicity. Surprisingly, these immunogenic “hot spot” regions are located throughout the human TGFb1 pre-protein, including within the SP and LAP domains, as well as the mature TGFb1 peptide. The present inventors have also identified a sub-region within the human TGFb1 LAP, i.e. positions 121-160 of SEQ ID NO: 1 (corresponding to the sequence of SEQ ID NO: 65), which harbours a greater frequency of immunogenic peptide sequences.

Thus, the present invention provides a polypeptide which is an immunogenic fragment of human TGFb1 (SEQ ID NO: 1) and which comprises or consists of a sequence of at least 9 consecutive amino acids of SEQ ID NO: 1. The sequence of at least 9 consecutive amino acids of SEQ ID NO: 1 may correspond to a sequence of at least 9 consecutive amino acids of SEQ ID NOs: 2 or 65. The polypeptide may comprise or consists of up to 9, 10, 11, 12, 13, 14, 15, 16, 17, 18, 19, 20, 21, 22, 23, 24, 25, 26, 27, 28, 29, 30, 35, 40, 45 or 50 consecutive amino acids of SEQ ID NO: 1. The polypeptide may comprise or consist of the amino acid sequence of any one of SEQ ID NOs: 6, 42, 12, 23, 28, 49, 55, 63, 7-9, 43-45, 13-15, 24-26, 29-31, 50-52, 56-58, 64, 65, 2, 66, 67, or 5, preferably the polypeptide comprises or consists of the amino acid sequence of any one of SEQ ID NOs: 6, 42, 12, 23, 28, 49, 55, 63, 66, 67 or 5. The polypeptide may comprise or consist of the amino acid sequence of any one of SEQ ID NOs: 66, 28-31, 67, 5-9, 42-45, 12-15, 55-58, 23-26, 49-52, 63, 64, 65 or 2, preferably the polypeptide comprises or consists of the amino acid sequence of any one of SEQ ID NOs: 66, 28, 67, 5, 6, 42, 12, 55, 23, 49, or 63.

The polypeptide may have a maximum length of 9, 10, 11, 12, 13, 14, 15, 16, 17, 18, 19, 20, 21, 22, 23, 24, 25, 26, 27, 28, 29, 30, 35, 40, 45 or 50 amino acids. The C terminal amino acid of the polypeptide may be replaced with the corresponding amide. The polypeptide may comprise a HLA-A2 restricted epitope. The HLA-A2-restricted epitope may comprise or consist of the amino acid sequence of SEQ ID NO: 66 or 67.

The present invention further provides a polynucleotide encoding a polypeptide of the invention. The polynucleotide may be isolated. A vector comprising the polynucleotide is also provided by the present invention.

The present invention also provides a composition comprising a polypeptide of the invention and/or a polynucleotide of the invention and optionally an adjuvant. The composition may further comprise at least one different polypeptide of the invention; at least one different polynucleotide of the invention; and/or at least one pharmaceutically acceptable diluent, carrier or preservative. The adjuvant may be selected from the group consisting of bacterial DNA based adjuvants, oil/surfactant based adjuvants, viral dsRNA based adjuvants, imidazochinilines, and a Montanide ISA adjuvant.

The present invention also provides a method of treating or preventing a disease or condition in a subject, the method comprising administering to the subject a polypeptide of the invention, a polynucleotide of the invention, and/or a composition of the invention. The method may further comprise the simultaneous or sequential administration of an additional cancer therapy, preferably an antibody.

The present invention also provides a polypeptide of the invention, a polynucleotide of the invention, a composition of the invention, or a combination thereof for use in treating or preventing a disease or condition. The polypeptide, polynucleotide, composition, or combination thereof may be for use in combination with an additional cancer therapy, preferably an antibody.

The present invention further provides use of a polypeptide of the invention, a polynucleotide of the invention, a composition of the invention, or a combination thereof for the manufacture of a medicament for the treatment or prevention of a disease or condition.

The disease or condition may be characterized at least in part by inappropriate or excessive immune suppressive function of TGFb 1-expressing cells, and/or wherein the disease or condition is cancer. The disease or condition may be characterized at least in part by inappropriate or excessive expression of interleukin-4 (IL-4) and/or interleukin 13 (IL-13). The disease or condition may be a cancer. Said cancer may be a breast cancer, a cervical cancer, a gastric cancer, a liver cancer, an ovarian cancer, a pancreatic cancer, lung cancer (such as a non-small-cell lung carcinoma (NSCLC)), a melanoma, a leukemia (such as an acute myeloid leukemia (AML)), or a prostate cancer.

The present invention further provides a method of stimulating TGFb1-specific T cells, the method comprising contacting the T cells with a polypeptide of the invention and/or a composition of the invention which comprises at least one polypeptide of the invention. The T cells may be present in a sample taken from a healthy subject or from a cancer patient, optionally a tumour sample.

### BRIEF DESCRIPTION OF THE FIGURES

FIG. 1A-C. Peptide-specific immune responses in PBMCs from 6 healthy donors were assessed against the array of 38 overlapping 20mer peptides derived from TGFb1 pre-protein by in vitro IFNγ ELISPOT assay, set up in triplicate wells. Each spot represents the average number of IFNγ-secreting cells after subtraction of the respective background signal, and the grey horizontal bars indicate the mean values across the tested donors. Stars indicate the peptides that elicited the strongest and the most DFRx2-based statistically significant responses and which were selected for further screening experiments (summarised in FIG. 1D).

FIG. 1D. A table summarising the most immunogenic TGFβ peptides and their respective mean IFNγ ELISPOT counts based on the screenings of FIGS. 1A-1C. The top eight best-performing peptides were selected for further investigation.

FIG. 1E. Top: Primary sequence of TGFb1 pre-protein. Highlighted are the amino acid sequences of the eight immunogenic TGFb1 peptides selected for further screening. Underlined amino acid position 1-29 indicate location of the signal sequence of the protein (SP), whereas underlined amino acid position 279-390 indicate the mature TGFb1 monomeric protein. Bottom: schematic representation of the TGFb1 pre-protein domains and the location of the eight selected TGFb1-derived peptides. The numbers (1, 29, 279 and 390) indicate key amino acid positions that flag the three main domains of the TGFb1 pre-protein.

FIG. 2. A. Peptide-specific immune responses in PBMCs against the eight immunogenic TGFb1-derived peptides identified in FIG. 1A-C were validated by assessing the responses in additional healthy donors by in vitro IFNγ ELISPOT assay. Each spot represents the average number of IFNγ-secreting cells in individual donor after subtraction of the respective background signal, and the black horizontal bars indicate the mean values across the tested donors. B. Heatmap depicting the amplitude of responses in PBMCs from healthy subject against lead epitopes (top); representative ELISPOT responses (bottom).

FIG. 3. A. Peptide-specific immune responses in PBMCs against the eight immunogenic TGFb1 peptides identified in FIG. 1A-C were validated, but this time examining cancer patients, by assessing the responses by in vitro IFNγ ELISPOT assay. Each spot represents the average number of IFNγ-secreting cells in individual cancer patient after subtraction of the respective background signal, and the black horizontal bars indicate the mean values across the tested patients. B. Heatmap depicting the amplitude of responses in PBMCs from cancer patients against lead epitopes.

FIG. 4. Intracellular Cytokine Staining (ICS) analysis was set up to further characterise the functionality of T cells responding to TGFb1 epitopes. In this example, the PBMCs from a healthy donor (BC-M-41) were thawed and stimulated with TGFb-02 (SEQ ID NO: 6), 13 days prior to the assay. IL-2 was added one day after the culture was set up (at 120 U/mL) and three days before the ICS was set up (at 60 U/mL). In each flow cytometry plot, each cell is represented as a dot, and the functional phenotype of the cells is analysed based on the expression of two markers at a time, one at each axis. Live cell populations were gated based on CD3⁺CD4⁺ T cell fractions or CD3⁺CD8⁺ T cell fractions and the expression of cytokine expression (IFNγ and TNFα) as well as marker for cytotoxicity (CD107a) were quantified. The percentages of the respective populations are summarised in the hierarchy table on the right.

FIG. 5. A. FACS plots showing CD4⁺ T-cell responses against TGFβ epitopes determined using ICS. B. FACS plots showing CD8⁺T-cell responses against TGFb1 epitopes determined using ICS.

FIG. 6. Bulk cultures specific for several TGFb1-derived epitopes were generated by MACS CD137 enrichment of specific T cells. The enriched cells were expanded after enrichment and showed variable reactivity against their epitope. For each of A-D, the top FACS plot show the amount of specific CD4⁺ gated cells and bottom FACS plot shows amount of specific CD8⁺ gate cells against the following epitopes: TGFb-02 (A), TGFb-05 (B), TGFb-26 (C), and TGFb-38 (D).

FIG. 7. A. Left: amplitude of responses in PBMCs from both cancer patients and healthy subjects measured by ex vivo ELISPOT. PBMCs were rested overnight and then plated directly in the ELISPOT wells and stimulated with epitope for 48 hours in the ELISPOT well. Right: examples of ex vivo ELISPOT responses against several of the TGFb1 lead epitopes. B. CD8⁺ T-cell responses identified against the epitope TGFb-15 after only 5 hours of stimulation using ICS.

FIG. 8. A. PBMCs from a patient with prostate cancer displaying a CD8⁺ T-cell response against the TGFb-15 epitope after 18-h stimulation with the epitope with a prior 14-day in vitro stimulation with the peptide. B. TGFb15-specific T cells from donor UR1121.14 were enriched twice after stimulation with TGFb-15, re-stimulated after 14 days of in vitro culture, and then enriched the next day using the MACS CD137 enrichment method. Both CD4⁺ T cells (top FACS plots for each of A and B) and CD8⁺ T cells (bottom FACS plots for each of A and B) responded to stimulation with TGFb-15.

FIG. 9. FACS plots showing the results of ICS analysis of TGFb-15-specific CD8⁺ T cell clones stimulated with TGFb-15.

FIG. 10. TGFb-15-specific CD8⁺ T cell clones kill target cells in an HLA-restricted manner and kill cancer cell lines expressing TGFb1. A. TGFb-15-specific CD8⁺ T cells effectively lysed T2 cells pulsed with TGFb-15 peptide. B. To ensure that the TGFb-15 response was HLA-A2 restricted, it was demonstrated that HLA-A2⁺ target cells but not HLA-A3⁺ target cells pulsed with peptide were lysed. C. Stimulation of clones with the HLA-A2⁺ cancer cell lines UKE-1 and THP-1 activated the TGFb-15-specific CD8⁺ T cell clones. Other HLA-A2⁺ cancer cells did not activate the T cells. D. Both THP-1 and UKE-1 cancer cell lines were readily killed by the TGFb-15-specific T cells. E. Activation of the TGFb-15-specific T cells was enhanced upon stimulation with cytokine-treated THP-1 cells. F. Stimulation of THP-1 cells with the Th2 cytokine IL-4 or with TGFb1 enhanced the amount of THP-1 cells killed by the TGFb-15-specific cells.

FIG. 11. The results of IFN-γ (A) and TNF-α (B) ELISPOT assays used to analyze responses against the TGF nonamer library spanning

FIG. 12. CD8⁺ T cells specific for an HLA-A2 binding decamer epitope in the TGFb1 signal peptide sequence readily kill TGFb1-expressing cancer cell lines in an HLA-A2-restricted manner. A. Healthy donor PBMCs displayed secreted IFN-γ upon stimulation with the HLA-A2-binding decamer epitope TGFb-A2-01 peptide after 14 days of in vitro culture. B. Intracellular cytokine staining of healthy donor PBMCs showed a CD8⁺ T-cell response against TGFb-A2-01 as stimulated CD8⁺ cells showed both enhanced IFN-γ and TFN-α expression (left) in addition to enhanced CD107a expression (right) upon stimulation with TGFb-A2-01. C. TGFb-A2-01-specific CD8⁺ T cells from a healthy donor killed TGFb-A2-01-pulsed HLA-A2⁺ target cells, whereas un-pulsed cells and peptide-pulsed HLA-A3⁺ target cells were not killed. D. The CD8⁺ T cells killed HLA-A2⁺ TGFb1— expressing UKE-1 target cells, whereas MARIMO and WM852 cells were not killed. E. HLA-A2⁺ THP-1 cells were readily killed by the TGFb-A2-01-specific T cells, and modulation of TGFb1 expression of the THP-1 cells by stimulation with different cytokines 48 hours before assaying the enhanced fraction of killed target cells.

FIG. 13. FACS plots showing the results of ICS analysis of TGFb-A2-01-specific T cell clones stimulated with TGFb-A2-01.

FIG. 14. Amino acid sequences of 20mer peptides in the TGFβ library. Overlapping amino acid sequences are underlined.

### BRIEF DESCRIPTION OF THE SEQUENCES

SEQ ID NO: 1 is the amino acid sequence of the full-length precursor of human TGFb1 (also referred to as the TGFb1 pre-protein).

SEQ ID NO: 2 is the amino acid sequence of the signal peptide of human TGFb1.

SEQ ID NO: 3 is the amino acid sequence of the LAP peptide of human TGFb1.

SEQ ID NO: 4 is the amino acid sequence of mature human TGFb1.

SEQ ID NOs: 5-64 are each an amino acid sequence of a polypeptide fragment derived from human TGFb1.

SEQ ID NO: 65 is the amino acid sequence of the LAP sub-region comprising a high frequency of immunogenic sequences.

SEQ ID NO: 66 is the amino acid sequence of the minimal epitope sequence within the TGFb-15 peptide sequence (SEQ ID NO: 28). SEQ ID NO: 66 is also referred to herein as “TGFb-15short”.

SEQ ID NO: 67 is the amino acid sequence of TGFb-A2-01.

### DETAILED DESCRIPTION OF THE INVENTION

It is to be understood that different applications of the disclosed products and methods may be tailored to the specific needs in the art. It is also to be understood that the terminology used herein is for the purpose of describing particular embodiments of the invention only, and is not intended to be limiting.

In addition as used in this specification and the appended claims, the singular forms “a”, “an”, and “the” include plural referents unless the content clearly dictates otherwise. Thus, for example, reference to “a polypeptide” includes “polypeptides”, and the like.

A “polypeptide” is used herein in its broadest sense to refer to a compound of two or more subunit amino acids, amino acid analogs, or other peptidomimetics. The term “polypeptide” thus includes short peptide sequences and also longer polypeptides and proteins. As used herein, the term “amino acid” refers to either natural and/or unnatural or synthetic amino acids, including both D or L optical isomers, and amino acid analogs and peptidomimetics.

The terms “patient” and “subject” are used interchangeably and typically refer to a human.

All publications, patents and patent applications cited herein, whether supra or infra, are hereby incorporated by reference in their entirety.

By “immunogenic” herein it is meant that a polypeptide is capable of eliciting an immune response to the TGFb1 protein, preferably when said protein is present in or on cells expressing the TGFb1 protein. In other words, the polypeptide may be described as immunogenic to TGFb1. The polypeptide may alternatively be described as an immunogenic fragment of TGFb1. The immune response is preferably a T cell response, and so the polypeptide may be described as an immunogenic fragment of TGFb1 comprising a T cell epitope. The immune response may be detected in at least one individual (or in sample taken from the individual) after administration of the polypeptide to said individual (or said sample).

A polypeptide may be identified as immunogenic using any suitable method, including in vitro methods. For example, a peptide may be identified as immunogenic if it has at least one of the following characteristics:
i. it is capable of eliciting IFN-γ-producing cells in a PBL population of a healthy subject and/or a cancer patient as determined by an ELISPOT assay; and/or
ii. it is capable of in situ detection in a sample of tumor tissue of CTLs that are reactive with TGFb1; and/or
iii. it is capable of inducing the in vitro growth of specific T-cells.
   Methods suitable for determining whether a polypeptide is immunogenic are also described in the Examples section below.

The polypeptide of the invention is an immunogenic fragment of human TGFb1 (SEQ ID NO: 1) which comprises or consists of a sequence of at least 9 consecutive amino acids of SEQ ID NO: 1.

The sequence of at least 9 consecutive amino acids of SEQ ID NO: 1 may correspond to a sequence of at least 9 consecutive amino acids of the SP domain of TGFb1, for example a sequence of at least 95 consecutive amino acids of SEQ ID NO: 2.

The sequence of at least 9 consecutive amino acids of SEQ ID NO: 1 may correspond to a sequence of at least 9 amino acids of the LAP domain of TGFb1, for example at least 9 consecutive amino acids of SEQ ID NO: 3.

The sequence of at least 9 consecutive amino acids of SEQ ID NO: 1 may correspond to a sequence of at least 9 consecutive amino acids located within the LAP sub-region bounded by amino acid positions 121 and 160 of SEQ ID NO: 1, for example a sequence of at least 9 consecutive amino acids of SEQ ID NO: 65.

The sequence of at least 9 consecutive amino acids of SEQ ID NO: 1 may correspond to a sequence of at least 9 consecutive amino acids of the mature TGFb1 polypeptide, for example a sequence of at least 9 consecutive amino acids of SEQ ID NO: 4.

The polypeptide may comprise or consist of up to 9, 10, 11, 12, 13, 14, 15, 16, 17, 18, 19, 20, 21, 22, 23, 24, 25, 26, 27, 28, 29, 30, 35, 40, 45 or 50 consecutive amino acids of SEQ ID NO: 1.

The polypeptide may comprise or consist of the amino acid sequence of any one of SEQ ID NOs: 2 and 5-67.

The polypeptide may comprise or consist of the amino acid sequence of any one of SEQ ID NOs: 6, 42, 12, 23, 28, 49, 55, 63, 5, 7-9, 43-45, 13-15, 24-26, 29-31, 50-52, 56-58, 64, 65, 2, 66, 6 or 5. Preferred are polypeptides that comprise or consist of the amino acid sequence of any one of SEQ ID NOs: 6, 42, 12, 23, 28, 49, 55, 63, 66, 67 or 5.

The polypeptide may comprise or consist of the amino acid sequence of any one of SEQ ID NOs: 66, 28-31, 67, 5-9, 42-45, 12-15, 55-58, 23-26, 49-52, 63, 64, 65 or 2. Particularly preferred are polypeptides that comprise or consist of the amino acid sequence of SEQ ID NOs: 66, 28, 67, 5, 6, 42, 12, 55, 23, 49 or 63.

The polypeptide may have a maximum length of 9, 10, 11, 12, 13, 14, 15, 16, 17, 18, 19, 20, 21, 22, 23, 24, 25, 26, 27, 28, 29, 30, 35, 40, 45 or 50 amino acids. The C terminal amino acid of the polypeptide may be replaced with the corresponding amide. The polypeptide may be isolated.

Particularly preferred polypeptides comprise or consist of the amino acid sequence of any one of SEQ ID NOs: 6, 42, 12, 23, 28, 49, 55, or 63. Particularly preferred polypeptides comprise or consist of the amino acid sequence of any one of SEQ ID NOs: 66, 28, 67, 5, 6, 42, 12, 55, 23, 49 or 63. Longer polypeptide fragments of SEQ ID NO: 1 which incorporate these sequences are also preferred.

The polypeptide may comprise a HLA-A2 restricted epitope. Preferably, the HLA-A2-restricted epitope comprises or consists of the amino acid sequence of SEQ ID NO: 66. Preferred peptides which comprise a HLA-A2 restricted epitope consisting of the amino acid sequence of SEQ ID NO: 66 are peptides which comprise or consist of the amino acid sequence of any one of SEQ ID NOs: 28-31 or 65. Alternatively, the HLA-A2-restricted epitope preferably comprises or consists of the amino acid sequence of SEQ ID NO: 67. Preferred peptides which comprise a HLA-A2 restricted epitope consisting of the amino acid sequence of SEQ ID NO: 67 are peptides which comprise or consist of the amino acid sequences of any one of SEQ ID NOs: 5, 8, 9 or 2.

In any polypeptide described herein, the amino acid sequence may be modified by one, two, three, four, or five (that is up to five) additions, deletions or substitutions, provided that a polypeptide having the modified sequence exhibits the same or increased immunogenicity to TGFb1, as compared to a polypeptide having the unmodified sequence. By “the same” it is to be understood that the polypeptide of the modified sequence does not exhibit significantly reduced immunogenicity to TGFb1 as compared to polypeptide of the unmodified sequence. Any comparison of immunogenicity between sequences is to be conducted using the same assay. Unless otherwise specified, modifications to a polypeptide sequence are preferably conservative amino acid substitutions. Conservative substitutions replace amino acids with other amino acids of similar chemical structure, similar chemical properties or similar side-chain volume. The amino acids introduced may have similar polarity, hydrophilicity, hydrophobicity, basicity, acidity, neutrality or charge to the amino acids they replace. Alternatively, the conservative substitution may introduce another amino acid that is aromatic or aliphatic in the place of a pre-existing aromatic or aliphatic amino acid. Conservative amino acid changes are well-known in the art and may be selected in accordance with the properties of the 20 main amino acids as defined in Table A1 below. Where amino acids have similar polarity, this can be determined by reference to the hydropathy scale for amino acid side chains in Table A2.

TABLE A1
Chemical properties of amino acids

| | | | |
|---|---|---|---|
| Ala (A) | aliphatic, hydrophobic, neutral | Met (M) | hydrophobic, neutral |
| Cys (C) | polar, hydrophobic, neutral | Asn (N) | polar, hydrophilic, neutral |
| Asp (D) | polar, hydrophilic, charged (−) | Pro (P) | hydrophobic, neutral |
| Glu (E) | polar, hydrophilic, charged (−) | Gln (Q) | polar, hydrophilic, neutral |
| Phe (F) | aromatic, hydrophobic, neutral | Arg (R) | polar, hydrophilic, charged (+) |
| Gly (G) | aliphatic, neutral | Ser (S) | polar, hydrophilic, neutral |
| His (H) | aromatic, polar, hydrophilic, charged (+) | Thr (T) | polar, hydrophilic, neutral |
| Ile (I) | aliphatic, hydrophobic, neutral | Val (V) | aliphatic, hydrophobic, neutral |
| Lys (K) | polar, hydrophilic, charged(+) | Trp (W) | aromatic, hydrophobic, neutral |
| Leu (L) | aliphatic, hydrophobic, neutral | Tyr (Y) | aromatic, polar, hydrophobic |

TABLE A2
Hydropathy scale

| Side Chain | Hydropathy |
|---|---|
| Ile | 4.5 |
| Val | 4.2 |
| Leu | 3.8 |
| Phe | 2.8 |
| Cys | 2.5 |
| Met | 1.9 |
| Ala | 1.8 |
| Gly | −0.4 |
| Thr | −0.7 |
| Ser | −0.8 |
| Trp | −0.9 |
| Tyr | −1.3 |
| Pro | −1.6 |
| His | −3.2 |
| Glu | −3.5 |
| Gln | −3.5 |
| Asp | −3.5 |
| Asn | −3.5 |
| Lys | −3.9 |
| Arg | −4.5 |

In any polypeptide disclosed herein, any one or more of the following modifications may be made to improve physiochemical properties (e.g. stability), provided that the polypeptide exhibits the same or increased immunogenicity to TGFb1, as compared to a polypeptide having the unmodified sequence:

Replacement of the C terminal amino acid with the corresponding amide (may increase resistance to carboxypeptidases);

Replacement of the N terminal amino acid with the corresponding acylated amino acid (may increase resistance to aminopeptidases);

Replacement of one or more amino acids with the corresponding methylated amino acids (may improve proteolytic resistance); and/or

Replacement of one or more amino acids with the corresponding amino acid in D-configuration (may improve proteolytic resistance).

Any polypeptide disclosed herein may have attached at the N and/or C terminus at least one additional moiety to improve solubility, stability and/or to aid with manufacture/isolation, provided that the polypeptide exhibits the same or increased immunogenicity to TGFb1, as compared to a polypeptide lacking the additional moiety. Suitable moieties include hydrophilic amino acids. For example, the amino acid sequences KK, KR or RR may be added at the N terminus and/or C terminus. Other suitable moieties include Albumin or PEG (Polyethylene Glycol).

A polypeptide as disclosed herein may be produced by any suitable means. For example, the polypeptide may be synthesised directly using standard techniques known in the art, such as Fmoc solid phase chemistry, Boc solid phase chemistry or by solution phase peptide synthesis. Alternatively, a polypeptide may be produced by transforming a cell, typically a bacterial cell, with a nucleic acid molecule or vector which encodes said polypeptide. The invention provides nucleic acid molecules and vectors which encode a polypeptide of the invention. The invention also provides a host cell comprising such a nucleic acid or vector.

The terms “polynucleotide” and “nucleic acid molecule” are used interchangeably herein and refer to a polymeric form of nucleotides of any length, either deoxyribonucleotides or ribonucleotides, or analogs thereof. Non-limiting examples of polynucleotides include a gene, a gene fragment, messenger RNA (mRNA), cDNA, recombinant polynucleotides, plasmids, vectors, isolated DNA of any sequence, isolated RNA of any sequence, nucleic acid probes, and primers. A polynucleotide of the invention may be provided in isolated or substantially isolated form. By substantially isolated, it is meant that there may be substantial, but not total, isolation of the polypeptide from any surrounding medium. The polynucleotides may be mixed with carriers or diluents which will not interfere with their intended use and still be regarded as substantially isolated. A nucleic acid sequence which “encodes” a selected polypeptide is a nucleic acid molecule which is transcribed (in the case of DNA) and translated (in the case of mRNA) into a polypeptide in vivo when placed under the control of appropriate regulatory sequences, for example in an expression vector. The boundaries of the coding sequence are determined by a start codon at the 5′ (amino) terminus and a translation stop codon at the 3′ (carboxy) terminus. For the purposes of the invention, such nucleic acid sequences can include, but are not limited to, cDNA from viral, prokaryotic or eukaryotic mRNA, genomic sequences from viral or prokaryotic DNA or RNA, and even synthetic DNA sequences. A transcription termination sequence may be located 3′ to the coding sequence.

Polynucleotides can be synthesised according to methods well known in the art, as described by way of example in Sambrook et al (1989, Molecular Cloning—a laboratory manual; Cold Spring Harbor Press). The nucleic acid molecules of the present invention may be provided in the form of an expression cassette which includes control sequences operably linked to the inserted sequence, thus allowing for expression of the polypeptide of the invention in vivo. These expression cassettes, in turn, are typically provided within vectors (e.g., plasmids or recombinant viral vectors). Such an expression cassette may be administered directly to a host subject. Alternatively, a vector comprising a polynucleotide of the invention may be administered to a host subject. Preferably the polynucleotide is prepared and/or administered using a genetic vector. A suitable vector may be any vector which is capable of carrying a sufficient amount of genetic information, and allowing expression of a polypeptide of the invention.

The present invention thus includes expression vectors that comprise such polynucleotide sequences. Such expression vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for expression of a peptide of the invention. Other suitable vectors would be apparent to persons skilled in the art. By way of further example in this regard we refer to Sambrook et al. (1989, Molecular Cloning—a laboratory manual; Cold Spring Harbor Press)

The invention also includes cells that have been modified to express a polypeptide of the invention. Such cells typically include prokaryotic cells such as bacterial cells, for example E. coli. Such cells may be cultured using routine methods to produce a polypeptide of the invention.

The polypeptide of the invention may be in a substantially isolated form. It may be mixed with carriers, preservatives, or diluents which will not interfere with the intended use, and/or with an adjuvant and still be regarded as substantially isolated. It may also be in a substantially purified form, in which case it will generally comprise at least 90%, e.g. at least 95%, 98% or 99%, of the protein in the preparation.

### Compositions Comprising Polypeptides

The present invention provides a composition comprising a polypeptide of the invention and/or a polynucleotide of the invention. For example, the invention provides a composition comprising one or more polypeptides of the invention and/or one or more polynucleotides of the invention, and optionally at least one adjuvant, pharmaceutically acceptable carrier, preservative and/or excipient.

The composition may comprise at least two, at least three, at least four, at least five, at least six, at least seven, at least eight different polypeptides of the invention and optionally at least one adjuvant, pharmaceutically acceptable carrier, preservative and/or excipient.

The composition may comprise at least two, at least three, at least four, at least five, at least six, at least seven, at least eight different polynucleotides of the invention and optionally at least one adjuvant, pharmaceutically acceptable carrier, preservative and/or excipient.

The carrier, preservative and excipient must be ‘acceptable’ in the sense of being compatible with the other ingredients of the composition and not deleterious to a subject to which the composition is administered. Typically, all components and the final composition are sterile and pyrogen free. The composition may be a pharmaceutical composition. The composition may preferably comprise an adjuvant. Adjuvants are any substance whose admixture into the composition increases or otherwise modifies the immune response elicited by the composition. Adjuvants, broadly defined, are substances which promote immune responses. Adjuvants may also preferably have a depot effect, in that they also result in a slow and sustained release of an active agent from the administration site. A general discussion of adjuvants is provided in Goding, Monoclonal Antibodies: Principles & Practice (2nd edition, 1986) at pages 61-63.

Adjuvants may be selected from the group consisting of: AlK(SO4)2, AlNa(SO4)2, Al NH4 (SO4), silica, alum, Al(OH)3, Ca3 (PO4)2, kaolin, carbon, aluminum hydroxide, muramyl dipeptides, N-acetyl-muramyl-L-threonyl-D-isoglutamine (thr-DMP), N-acetyl-nornuramyl-L-alanyl-D-isoglutamine (CGP 11687, also referred to as nor-MDP), N-acetylmuramyul-L-alanyl-D-isoglutaminyl-L-alanine-2-(1′2′-dipalmitoyl-sn-glycero-3-hydroxphosphoryloxy)-ethylamine (CGP 19835A, also referred to as MTP-PE), RIBI (MPL+TDM+CWS) in a 2% squalene/Tween-80® emulsion, lipopolysaccharides and its various derivatives, including lipid A, Freund's Complete Adjuvant (FCA), Freund's Incomplete Adjuvants, Merck Adjuvant 65, polynucleotides (for example, poly IC and poly AU acids), wax D from Mycobacterium, tuberculosis, substances found in Corynebacterium parvum, Bordetella pertussis, and members of the genus Brucella, Titermax, ISCOMS, Quil A, ALUN (see U.S. Pat. Nos. 58,767 and 5,554,372), Lipid A derivatives, choleratoxin derivatives, HSP derivatives, LPS derivatives, synthetic peptide matrixes or GMDP, Interleukin 1, Interleukin 2, Montanide ISA-51 and QS-21. Various saponin extracts have also been suggested to be useful as adjuvants in immunogenic compositions. Granulocyte-macrophage colony stimulating factor (GM-CSF) may also be used as an adjuvant.

Preferred adjuvants to be used with the invention include oil/surfactant based adjuvants such as Montanide adjuvants (available from Seppic, Belgium), preferably Montanide ISA-51. Other preferred adjuvants are bacterial DNA based adjuvants, such as adjuvants including CpG oligonucleotide sequences. Yet other preferred adjuvants are viral dsRNA based adjuvants, such as poly I:C. GM-CSF and Imidazochinilines are also examples of preferred adjuvants.

The adjuvant is most preferably a Montanide ISA adjuvant. The Montanide ISA adjuvant is preferably Montanide ISA 51 or Montanide ISA 720.

In Goding, Monoclonal Antibodies: Principles & Practice (2nd edition, 1986) at pages 61-63 it is also noted that, when an antigen of interest is of low molecular weight, or is poorly immunogenic, coupling to an immunogenic carrier is recommended. A polypeptide of the invention may therefore be coupled to a carrier. A carrier may be present independently of an adjuvant. The function of a carrier can be, for example, to increase the molecular weight of a polypeptide fragment in order to increase activity or immunogenicity, to confer stability, to increase the biological activity, or to increase serum half-life. Furthermore, a carrier may aid in presenting the polypeptide or fragment thereof to T-cells. Thus, in the composition, the polypeptide may be associated with a carrier such as those set out below. The carrier may be any suitable carrier known to a person skilled in the art, for example a protein or an antigen presenting cell, such as a dendritic cell (DC). Carrier proteins include keyhole limpet hemocyanin, serum proteins such as transferrin, bovine serum albumin, human serum albumin, thyroglobulin or ovalbumin, immunoglobulins, or hormones, such as insulin or palmitic acid. Alternatively the carrier protein may be tetanus toxoid or diphtheria toxoid. Alternatively, the carrier may be a dextran such as sepharose. The carrier must be physiologically acceptable to humans and safe.

If the composition comprises an excipient, it must be ‘pharmaceutically acceptable’ in the sense of being compatible with the other ingredients of the composition and not deleterious to the recipient thereof. Auxiliary substances, such as wetting or emulsifying agents, pH buffering substances and the like, may be present in the excipient. These excipients and auxiliary substances are generally pharmaceutical agents that do not induce an immune response in the individual receiving the composition, and which may be administered without undue toxicity. Pharmaceutically acceptable excipients include, but are not limited to, liquids such as water, saline, polyethyleneglycol, hyaluronic acid, glycerol and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients, vehicles and auxiliary substances is available in Remington's Pharmaceutical Sciences (Mack Pub. Co., N.J. 1991).

Formulation of a suitable composition can be carried out using standard pharmaceutical formulation chemistries and methodologies all of which are readily available to the reasonably skilled artisan. Such compositions may be prepared, packaged, or sold in a form suitable for bolus administration or for continuous administration. Injectable compositions may be prepared, packaged, or sold in unit dosage form, such as in ampoules or in multi-dose containers optionally containing a preservative. Compositions include, but are not limited to, suspensions, solutions, emulsions in oily or aqueous vehicles, pastes, and implantable sustained-release or biodegradable formulations. In one embodiment of a composition, the active ingredient is provided in dry (for e.g., a powder or granules) form for reconstitution with a suitable vehicle (e.g., sterile pyrogen-free water) prior to administration of the reconstituted composition. The composition may be prepared, packaged, or sold in the form of a sterile injectable aqueous or oily suspension or solution. This suspension or solution may be formulated according to the known art, and may comprise, in addition to the active ingredient, additional ingredients such as the adjuvants, excipients and auxiliary substances described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono-or di-glycerides. Other compositions which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt. Alternatively, the active ingredients of the composition may be encapsulated, adsorbed to, or associated with, particulate carriers. Suitable particulate carriers include those derived from polymethyl methacrylate polymers, as well as PLG microparticles derived from poly(lactides) and poly(lactide-co-glycolides). See, e.g., Jeffery et al. (1993) Pharm. Res. 10:362-368. Other particulate systems and polymers can also be used, for example, polymers such as polylysine, polyarginine, polyornithine, spermine, spermidine, as well as conjugates of these molecules.

### Methods of Use

The polypeptide, polynucleotide, or composition of the invention, or a combination thereof may be used in a method of treating or preventing a disease or condition in a subject. The polypeptide, polynucleotide or composition of the invention, or combination thereof may be used in the manufacture of a medicament for use in a method of treating or preventing a disease or condition in a subject. The method comprises administering to the said subject the said polypeptide, the said polynucleotide, the said composition, or the said combination. Administration may be of a therapeutically or prophylactically effective quantity of the said polypeptide, the said polynucleotide, the said composition, or the said combination to a subject in need thereof.

The disease or condition may be characterized at least in part by inappropriate or excessive immune suppressive function of TGFb 1. The disease or condition may be characterized at least in part by inappropriate or excessive expression of IL-4 and/or IL-13. The disease or condition may be a cancer, preferably a cancer which expresses TGFb1 and/or which is associated with inappropriate or excessive immune suppressive function of TGFb1 and or inappropriate or excessive expression of IL-4 and/or IL-13. The cancer may be breast, cervical, gastric, liver, ovarian or pancreatic cancer, lung cancer (such as non-small-cell lung carcinoma (NSCLC)), melanoma, leukemia (such as acute myeloid leukemia (AML)), or prostate cancer. The cancer may be AML characterized by inappropriate or excessive immune suppressive function of TGFb1 and/or inappropriate or excessive expression of IL-4 and/or IL-13. The cancer may be AML characterized by inappropriate or excessive immune suppressive function of TGFb1 and inappropriate or excessive expression of IL-4 and/or IL-13.

The method may comprise simultaneous or sequential administration with an additional cancer therapy. The additional cancer therapy may be a bi-specific inhibitor of TGFb (e.g. TGFb1) and PD-L1. Said bi-specific inhibitor may be capable of simultaneously binding to, and/or inhibiting the activity of, TGFb and PD-L1. Said bi-specific inhibitor may be a fusion protein comprising an anti-TGFb portion and an anti-PD-L1 portion, optionally wherein the anti-PD-L1 portion comprises or consists of anti-PD-L1 antibody and/or the anti-TGFb portion comprises or consists of a receptor for TGFb or a portion thereof, such as TGFb receptor II or portion thereof.

The additional cancer therapy may be selected from a cytokine therapy, a T-cell therapy, an NK therapy, an immune system checkpoint inhibitor, chemotherapy, radiotherapy, immunostimulating substances, gene therapy, or an antibody.

The antibody may be Abagovomab, Abciximab, Actoxumab, Adalimumab, Adecatumumab, Afelimomab, Afutuzumab, Alacizumab pegol, ALD518, Alemtuzumab, Alirocumab, Altumomab pentetate, Amatuximab, Anatumomab mafenatox, Anrukinzumab, Apolizumab, Arcitumomab, Aselizumab, Atinumab, Atlizumab (=tocilizumab), Atorolimumab, Bapineuzumab, Basiliximab, Bavituximab, Bectumomab, Belimumab, Benralizumab, Bertilimumab, Besilesomab, Bevacizumab, Bezlotoxumab, Biciromab, Bimagrumab, Bivatuzumab mertansine, Blinatumomab, Blosozumab, Brentuximab vedotin, Briakinumab, Brodalumab, Canakinumab, Cantuzumab mertansine, Cantuzumab ravtansine, Caplacizumab, Capromab pendetide, Carlumab, Catumaxomab, CC49, Cedelizumab, Certolizumab pegol, Cetuximab, Ch.14.18, Citatuzumab bogatox, Cixutumumab, Clazakizumab, Clenoliximab, Clivatuzumab tetraxetan, Conatumumab, Concizumab, Crenezumab, CR6261, Dacetuzumab, Daclizumab, Dalotuzumab, Daratumumab, Demcizumab, Denosumab, Detumomab, Dorlimomab aritox, Drozitumab, Duligotumab, Dupilumab, Dusigitumab, Ecromeximab, Eculizumab, Edobacomab, Edrecolomab, Efalizumab, Efungumab, Elotuzumab Elsilimomab, Enavatuzumab, Enlimomab pegol, Enokizumab, Enoticumab, Ensituximab, Epitumomab cituxetan, Epratuzumab, Erlizumab, Ertumaxomab, Etaracizumab, Etrolizumab, Evolocumab, Exbivirumab, Fanolesomab, Faralimomab Farletuzumab, Fasinumab, FBTA05, Felvizumab, Fezakinumab, Ficlatuzumab, Figitumumab, Flanvotumab, Fontolizumab, Foralumab, Foravirumab, Fresolimumab, Fulranumab, Futuximab, Galiximab, Ganitumab, Gantenerumab, Gavilimomab, Gemtuzumab ozogamicin, Gevokizumab, Girentuximab, Glembatumumab vedotin, Golimumab, Gomiliximab, GS6624, Ibalizumab, Ibritumomab tiuxetan, Icrucumab, Igovomab, Imciromab, Imgatuzumab, Inclacumab, Indatuximab ravtansine, Infliximab, Intetumumab, Inolimomab, Inotuzumab ozogamicin, Ipilimumab, Iratumumab, Itolizumab, Ixekizumab, Keliximab, Labetuzumab, Lampalizumab, Lebrikizumab, Lemalesomab, Lerdelimumab, Lexatumumab, Libivirumab, Ligelizumab, Lintuzumab, Lirilumab, Lodelcizumab, Lorvotuzumab mertansine, Lucatumumab, Lumiliximab, Mapatumumab, Maslimomab, Mavrilimumab, Matuzumab, Mepolizumab, Metelimumab, Milatuzumab, Minretumomab, Mitumomab, Mogamulizumab, Morolimumab, Motavizumab, Moxetumomab pasudotox, Muromonab-CD3, Nacolomab tafenatox, Namilumab, Naptumomab estafenatox, Narnatumab, Natalizumab, Nebacumab, Necitumumab, Nerelimomab, Nesvacumab, Nimotuzumab, Nivolumab, Nofetumomab merpentan, Obinutuzumab, Ocaratuzumab, Ocrelizumab, Odulimomab, Ofatumumab, Olaratumab, Olokizumab, Omalizumab, Onartuzumab, Oportuzumab monatox, Oregovomab, Orticumab, Otelixizumab, Oxelumab, Ozanezumab, Ozoralizumab, Pagibaximab, Palivizumab, Panitumumab, Panobacumab, Parsatuzumab, Pascolizumab, Pateclizumab, Patritumab, Pemtumomab, Perakizumab, Pertuzumab, Pexelizumab, Pidilizumab, Pinatuzumab vedotin, Pintumomab, Placulumab, Polatuzumab vedotin, Ponezumab, Priliximab, Pritoxaximab, Pritumumab, PRO 140, Quilizumab, Racotumomab, Radretumab, Rafivirumab, Ramucirumab, Ranibizumab, Raxibacumab, Regavirumab, Reslizumab, Rilotumumab, Rituximab, Robatumumab, Roledumab, Romosozumab, Rontalizumab, Rovelizumab, Ruplizumab, Samalizumab, Sarilumab, Satumomab pendetide, Secukinumab, Seribantumab, Setoxaximab, Sevirumab, Sibrotuzumab, Sifalimumab, Siltuximab, Simtuzumab, Siplizumab, Sirukumab, Solanezumab, Solitomab, Sonepcizumab, Sontuzumab, Stamulumab, Sulesomab, Suvizumab, Tabalumab, Tacatuzumab tetraxetan, Tadocizumab, Talizumab, Tanezumab, Taplitumomab paptox, Tefibazumab, Telimomab aritox, Tenatumomab, Teneliximab, Teplizumab, Teprotumumab, TGN1412, Ticilimumab (=tremelimumab), Tildrakizumab, Tigatuzumab, TNX-650, Tocilizumab (=atlizumab), Toralizumab, Tositumomab, Tralokinumab, Trastuzumab, TRBS07, Tregalizumab, Tremelimumab Tucotuzumab celmoleukin, Tuvirumab, Ublituximab, Urelumab, Urtoxazumab, Ustekinumab, Vapaliximab, Vatelizumab, Vedolizumab, Veltuzumab, Vepalimomab Vesencumab, Visilizumab, Volociximab, Vorsetuzumab mafodotin, Votumumab, Zalutumumab, Zanolimumab, Zatuximab, Ziralimumab or Zolimomab aritox. Preferred antibodies include Natalizumab, Vedolizumab, Belimumab, Atacicept, Alefacept, Otelixizumab, Teplizumab, Rituximab, Ofatumumab, Ocrelizumab, Epratuzumab, Alemtuzumab, Abatacept, Eculizumab, Omalizumab, Canakinumab, Meplizumab, Reslizumab, Tocilizumab, Ustekinumab, Briakinumab, Etanercept, Inlfliximab, Adalimumab, Certolizumab pegol, Golimumab, Trastuzumab, Gemtuzumab, Ozogamicin, Ibritumomab, Tiuxetan, Tostitumomab, Cetuximab, Bevacizumab, Panitumumab, Denosumab, Ipilimumab, Brentuximab and Vedotin.

Particularly preferred antibodies that may be used in the method of the invention include: daratumumab, nivolumab, pembrolizumab, avelumab, rituximab, trastuzumab, pertuzumab, alemtuzumab, cetuximab, panitumumab, tositumomab and of atumumab.

The additional cancer therapy may be selected from the group consisting of Actimide, Azacitidine, Azathioprine, Bleomycin, Carboplatin, Capecitabine, Cisplatin, Chlorambucil, Cyclophosphamide, Cytarabine, Dauno-rubicin, Docetaxel, Doxifluridine, Doxorubicin, Epirubicin, Etoposide, Fludarabine, Fluor-ouracil, Gemcitabine, Hydroxyurea, Idarubicin, Irinotecan, Lenalidomide, Leucovorin, Mechlorethamine, Melphalan, Mercaptopurine, Methotrexate, Mitoxantrone, Oxaliplatin, Paclitaxel, Pemetrexed, Revlimid, Temozolomide, Teniposide, Thioguanine, Valrubicin, Vinblastine, Vincristine, Vindesine and Vinorelbine.

A polypeptide of the invention and/or a composition of the invention comprising at least one polypeptide of the invention may also be used in a method of stimulating TGFb1-specific T cells, such as CD4⁺ and/or CD8⁺ T-cells, comprising contacting cells with the said polypeptide and/or said composition. The method may be conducted ex vivo. The cells may be present in a sample taken from a healthy subject or from a cancer patient, such as in a tumour sample.

The present invention is further illustrated by the following examples that, however, are not to be construed as limiting the scope of protection. The features disclosed in the foregoing description and in the following examples may, both separately and in any combination thereof, be material for realizing the invention in diverse forms thereof.

### EXAMPLES

### Example 1—Materials and Methods

### Patients and Donors

Buffy coats from anonymized blood donors were acquired from the blood bank at Rigshospitalet, Copenhagen, Denmark. Buffy coats from cancer patients were acquired from the Department of Oncology, Herlev Hospital, Herlev, Denmark. All participants provided informed consent before study entry, in agreement with the Helsinki declaration. PBMCs were isolated with Lymphoprep (Axis Shield, Oslo, Norway) and frozen in fetal calf serum with 10% dimethyl sulfoxide (DMSO; Sigma-Aldrich, St. Louis, Mo., USA).

### Peptides

Peptides were provided by Pepscan (Lelystadt, Netherlands) and dissolved in DMSO at a concentration of 10 mM. After identification of the TGFβ1 lead epitopes, these peptides were provided at a higher purity (>90%) by KJ Ross-Petersen (Klampenborg, Denmark). The sequences of the peptides used in these experiments are shown in the section entitled “SEQUENCES”). Peptides are described by SEQ ID NO, by name, or by reference to the start and end positions of each peptide sequence within the amino acid sequence of the full length precursor of human TGFb1. Each designation may be used interchangeably, as indicated in the table set out in the SEQUENCES section below. For example, the peptide of SEQ ID NO: 6 may alternatively be referred to by the name TGFb-02 (or TGFB02), or may alternatively be referred to as TGFb1₁₁₋₃₀ (given a start position of 11 and end position of 30). The intended reference in each case will be clear from the context.

### In Vitro ELISPOT Assay

For in vitro ELISPOT, PBMCs from cancer patients and healthy donors were pulsed with 20 μM of TGFβ-derived peptides (or with no peptide as a control) and 120 U/ml IL-2 in 24-well plates for 7-10 days before being used in an ELISPOT assay. The cells were placed in 96-well nitrocellulose ELISPOT plates (MultiScreen IP Filter Plate, MSIPN4W50; Millipore) pre-coated with IFNγ capture antibody (Mabtech). TGFβ peptides are added to a final concentration of 5 μM, control stimulation (DMSO, HIV or scrambled peptide) added to control wells and plates are incubated at 37° C. for 16-20 hours. After the incubation the cells were washed off and secondary biotinylated Ab (Mabtech, cat. 3420-6-1000) was added for 2 hours at room temperature. Unbound secondary antibody was washed off and streptavidin conjugated alkaline phosphatase (AP) (Mabtech, cat. 3310-10) was added for 1 hour at room temperature. Unbound conjugated enzyme was washed off and the assay was developed by adding BCIP/NBT substrate (Mabtech, cat. 3650-10). Developed ELISPOT plates were analysed on CTL ImmunoSpot S6 Ultimate-V analyzer using Immunospot software v5.1. Responses were reported as the difference between average numbers of spots in wells stimulated with TGFβ peptide and wells without added peptide. Unless otherwise stated, all experiments were performed with the in vitro IFN-γ ELISPOT assay, and all experiments were performed in triplicate. Statistical analysis was performed using the distribution free resampling (DFR) method and with the more conservative, DFR2x method, as described by Moodie et al. (Cancer Immunol Immunother 2010; 59: 1489-1501).

### Ex Vivo ELISPOT Assay

PBMCs from cancer patients or healthy donors are thawed and rested overnight in 24-well plate in X-VIVO media. (Optional: 1 μg/m1DNase I added). The following day cells are counted and transferred to 96-well nitrocellulose ELISPOT plates (MultiScreen IP Filter Plate, MSIPN4W50; Millipore) pre-coated with IFNγ capture antibody (Mabtech). TGFβ peptides are added to a final concentration of 5 μM, control stimulation (DMSO, HIV or scrambled peptide) added to control wells and plates incubated at 37° C. for 24-72 hours. Plate staining with secondary antibodies and development protocol follows the in vitro ELISPOT protocol above.

### Intracellular Cytokine Staining (ICS) and Fluorescence Activated Cell Sorting (FACS)

Intracellular staining of cell cultures was performed after PBMCs were stimulated with TGFβ-derived peptides (or incubated with no peptide as a control) for 5 hours in the presence of BD GolgiPlug™ (added after the first hour of peptide stimulation). CD107a-PE (cat. 555801, BD Biosciences) antibody is added in the beginning of the incubation. Stimulated cells were stained with fluorescently labelled antibodies for surface markers (CD3, CD4, CD8) and thereafter permeabilised by using a mixture of Fixation/Permeabilization concentrate and diluent (eBioscience, cat. 00-5123-43 and 00-5223-56), according to manufacturer's instructions. Permeabilised cells were then stained with fluorochrome-labelled antibodies for IFNγ and TNFα. Flow cytometry analysis was performed on a FACSCanto™ II (BD Biosciences). Antibodies used: IFNγ-APC (cat. 341117), TNFα-455 BV421 (cat. 562783), CD4-FITC (cat. 347413) or CD4-PerCP (cat. 345770), CD8-PerCP (cat. 345774) or CD8-FITC (cat. 345772), CD3-APC-H7 (cat. 560275) (all from BD Biosciences). Dead cells were stained with Fixable Viability Stain 510 (BD Biosciences, San Jose, Calif., USA). Another way of identifying activated T cells was by overnight stimulation of T cells with either antigen or target cells. After 18-24 hours of stimulation, cells were stained with the above-mentioned surface antigen specific antibodies and fixable viability stain in concert with staining with anti-CD107a-PE and anti-CD137-BV421 (BD Biosciences, San Jose, Calif., USA). Donor PBMCs were analyzed for HLA-A2 by staining with anti-HLA-A2-FITC (BD Biosciences, San Jose, CA, USA) with an appropriate isotype control.

### Rapid Expansion Protocol

In some experiments, T cells were expanded using rapid expansion protocol (REP) with allogeneic irradiated peripheral blood mononuclear cells (PBMC) from at least three different healthy donors, 30 ng/mL anti-CD3 antibodies (OKT3, from Janssen-Cilag or Miltenyi Biotec) and high doses of IL-2 (6,000 IU/mL IL2; Proleukin from Novartis).

### FACS of Live Cells

For enrichment of specific T cells from a primary PBMC culture, the in vitro culture method for cell cultures destined for analysis in ELISPOT was followed (see above). Next, cells were stimulated with antigen overnight and the following day were washed twice in FACS buffer, then stained for 30 minutes with the following: LIVE/DEAD Fixable Near-IR Dead Cell Stain Kit (Waltham, Mass., USA), anti-CD4-FITC, anti-CD8-PerCP, anti-CD107a-PE and anti-CD137-BV421 (BD Biosciences, San Jose, Calif., USA). Cells were then washed twice and resuspended in FACS buffer. Next, cells were sorted on a FACS ARIA flow cytometer with appropriate application settings and compensation controls. Cell sorting was performed with a purity setting. After sorting, cells were split into two fractions—half of the enriched cells were expanded using a rapid expansion protocol, and the other half of the cells were cloned using limiting dilution with a seeding of three cells/well. Cloned cells were expanded using a rapid expansion protocol.

### Magnetically Activated Cell Sorting (MACS)

MACS was used to enrich for antigen-specific T cells both from primary cultures and from already enriched cultures. Enrichment of specific T cells from a primary PBMC culture followed the in vitro culture method for cell cultures destined for analysis in ELISPOT (see above). Next, cells were stimulated with antigen overnight and enriched the following day using the MACS CD137 enrichment kit (Miltenyi Biotech, Bergisch Gladbach, Germany), according to the manufacturer's protocol. Enriched cells were expanded using a rapid expansion protocol. Where stated, some of the enriched cells were cloned by limiting dilution. Cloned cells were expanded using a rapid expansion protocol.

### Chromium-51 Cytotoxicity Assay and CYTOKINE stimulation of Target Cells

A chromium-51 cytotoxicity assay was used to assess the killing potential of the specific T cells, as described in Andersen M H et al. (J Immunol 1999; 163: 3812-3818). To manipulate the expression of TGFβ in several cancer cell lines, the cancer cell lines were stimulated with IL-4 (100 U/mL), IL-13 (20 U/mL), and TGFβ1 (2.e5 ng/mL) (all Peprotech, Rocky Hill, N.J., USA) either alone or in combination for 48 hours before assaying.

### Example 2—In Vitro ELISPOT Screening of 20 mer Peptides

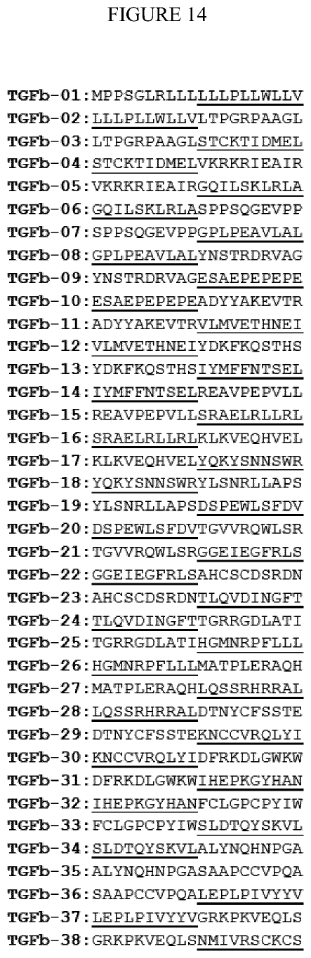

An array of 38 overlapping 20 mer peptides derived from the full-length TGFb1 precursor was designed and produced as described above. Each of the 20 mer peptides overlaps by 10 amino acids (see FIG. 14).

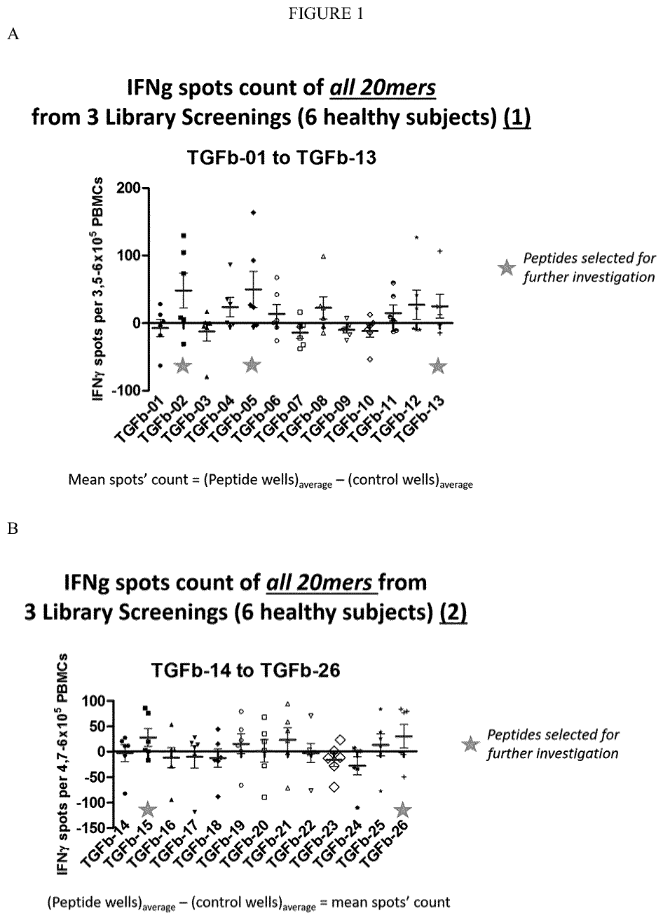

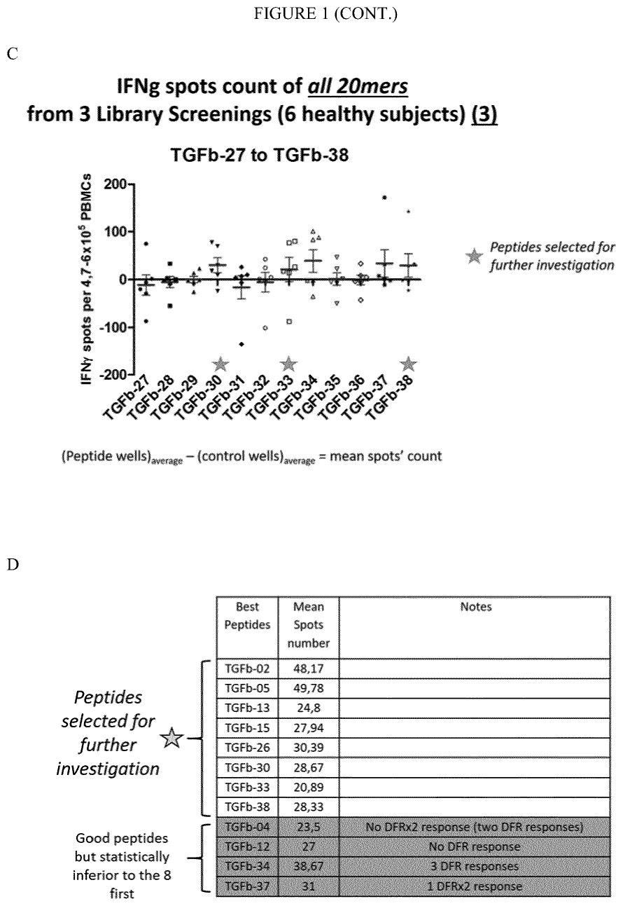

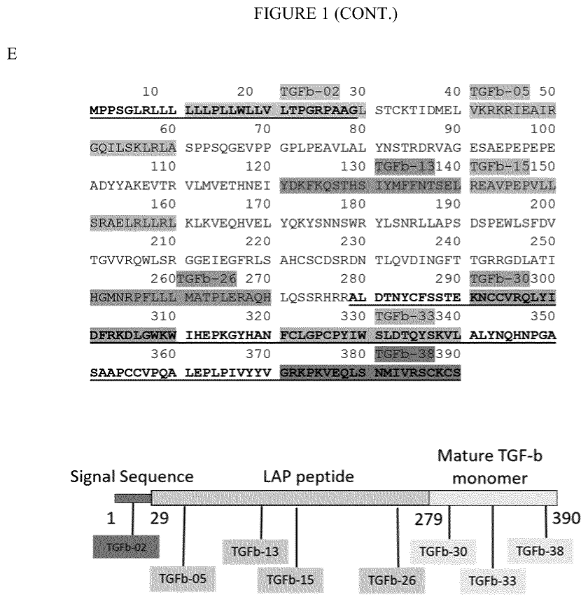

Peptide-specific immune responses in PBMCs from six healthy donors were assessed for spontaneous immune responses against the array of 20 mer peptides using in vitro IFNγ ELISPOT assay, set up in triplicate wells. The results of these assays are shown in FIGS. 1A-C. The peptides that elicited the strongest and the most statistically significant responses were selected for further screening experiments. The identity of the best-performing peptides is summarized in FIG. 1D and the positions of said peptides within the full-length sequence are indicated in FIG. 1E.

Surprisingly, it was observed that immunogenic peptides were located throughout the full-length sequence of the TGFb1 precursor protein, rather than being clustered within a single immunogenic ‘hot-spot’ or being located within the amino acid sequence of the mature TGFb1 peptide. Notably, immunogenic epitope peptides were identified with the signal peptide region of the TGFb1 precursor and the LAP peptide, which are not present in the mature, active form of TGFb1. In addition, an LAP sub-region with a high frequency of immunogenic peptides was identified, namely amino acids 121-160 of SEQ ID NO: 1 (corresponding to SEQ ID NO: 65). This region comprises the immunogenic peptides: TGFb-13 and TGFb-15.

The eight most immunogenic peptides, namely: TGFb-02, TGFb-26, TGFb-05, TGFb-13, TGFb-15, TGFb-30, TGFb-33, and TGFb-38 (corresponding to SEQ ID NOs: 6, 42, 12, 23, 28, 49, 55, and 63, respectively) were selected for further investigation.

### Example 3—Validation of Peptide-Specific Immune Responses

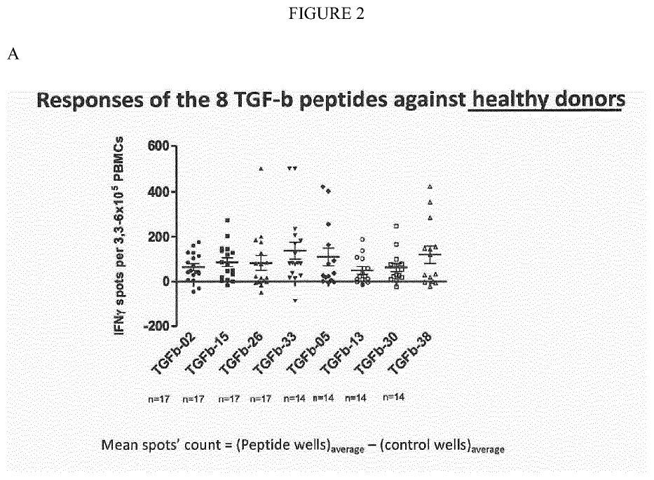

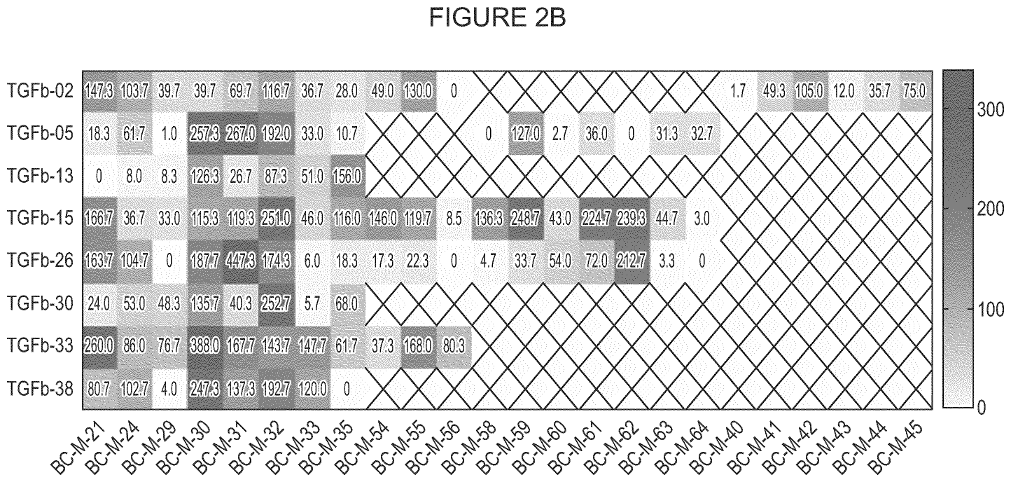

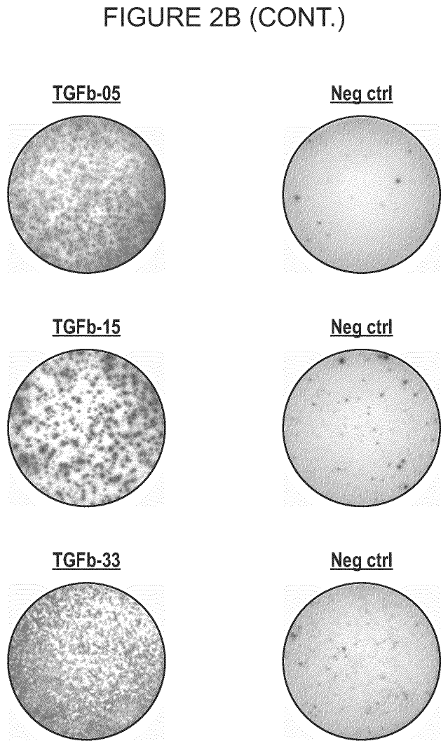

Additional in vitro IFNγ ELISPOT assays were set up to validate the responses against the eight selected epitope peptides identified in the initial screen (see Example 2) in additional healthy subjects. The results of these assays are presented in FIG. 2. Strong and frequent responses against all of the tested epitope peptides, with TGFb-02, TGFb-26, TGFb-33, and especially TGFb-15 showing strong and frequent responses (FIG. 2B).

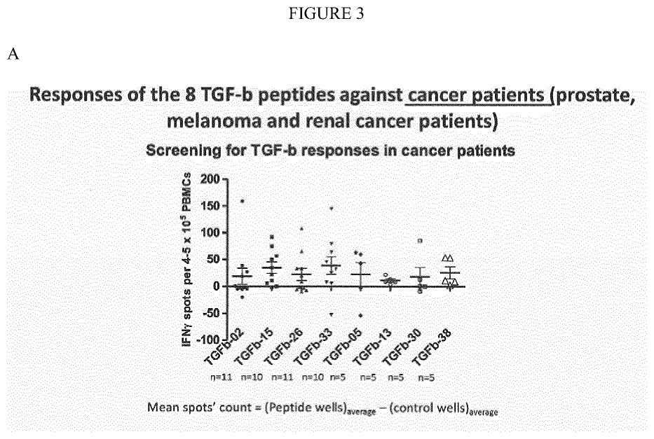

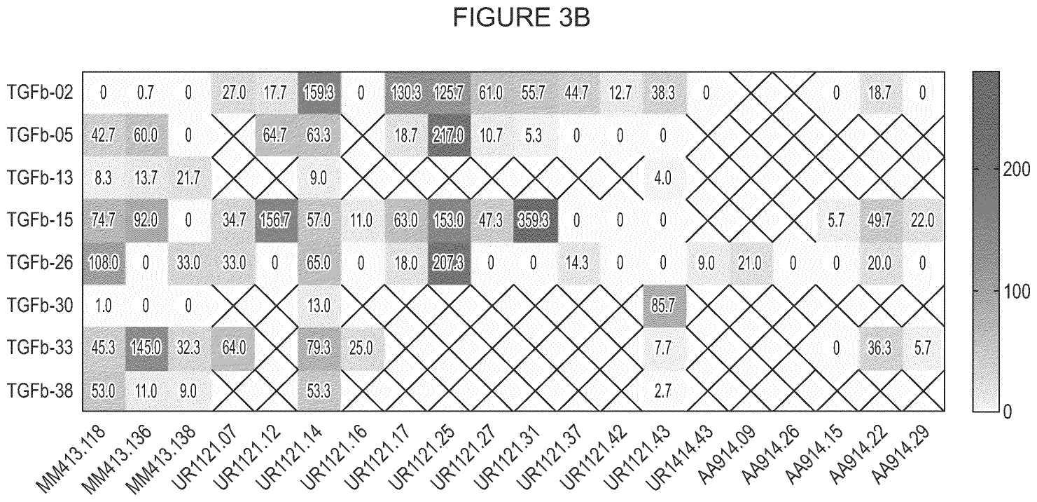

Healthy subjects and cancer patients can show different patterns of immune responses to epitopes, so the immunogenic potential of the selected epitopes in cancer patients was also investigated. Peptide-specific immune responses in PBMCs against the eight immunogenic TGFb1-derived peptides were also validated by examining cancer patients, again by assessing the responses by in vitro IFNγ ELISPOT assay. The results of these assays are presented in FIG. 3. TGFb-02, TGFb-15, TGFb-26, and TGFb-33 were observed to be highly immunogenic in patients (FIG. 3B).

### Example 4—Cytokine Analysis

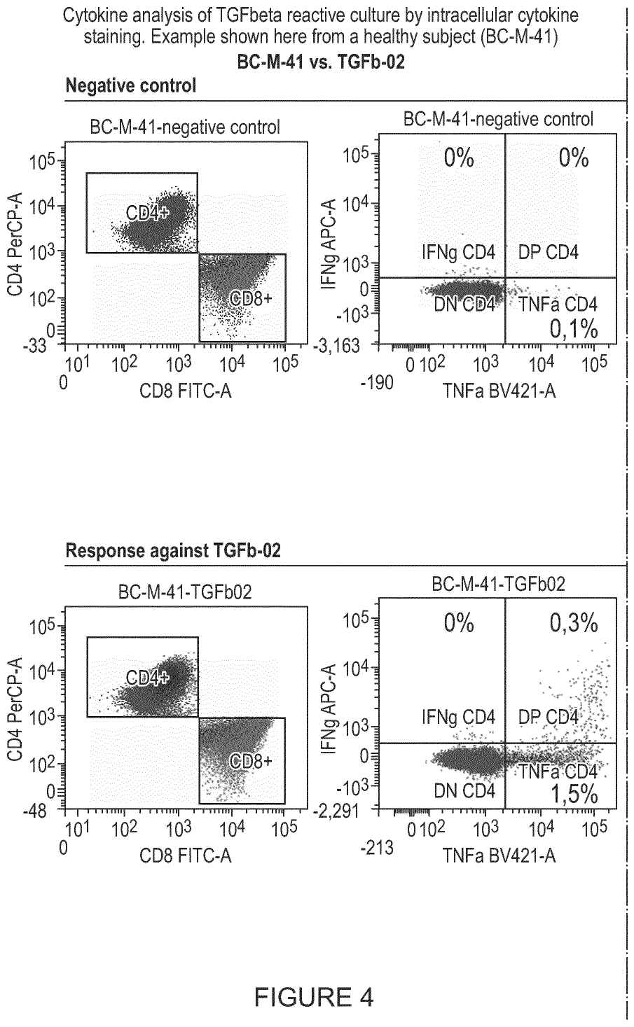

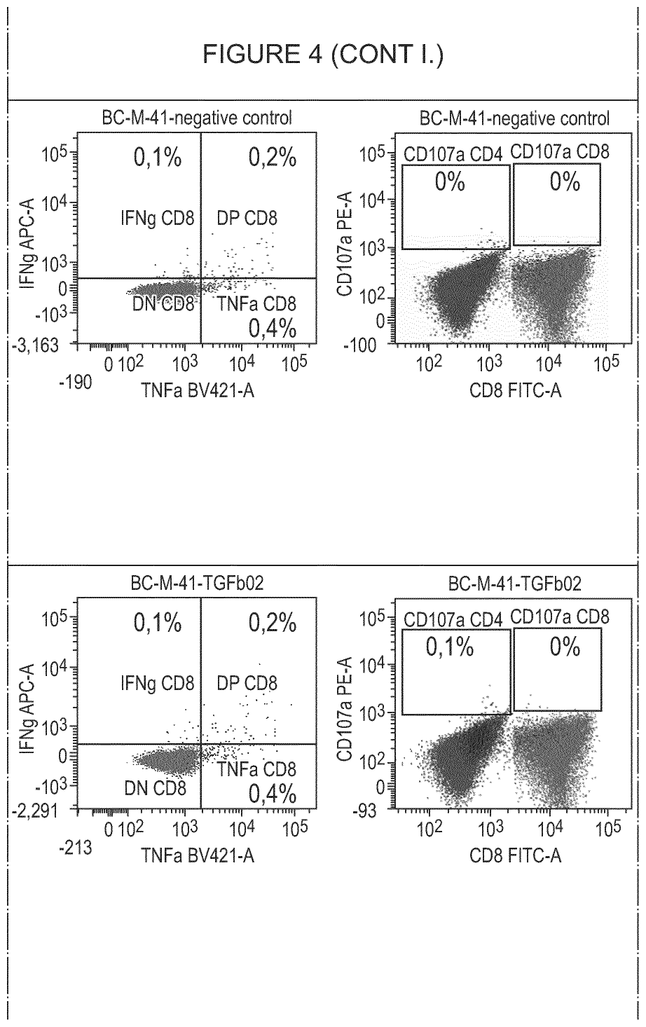

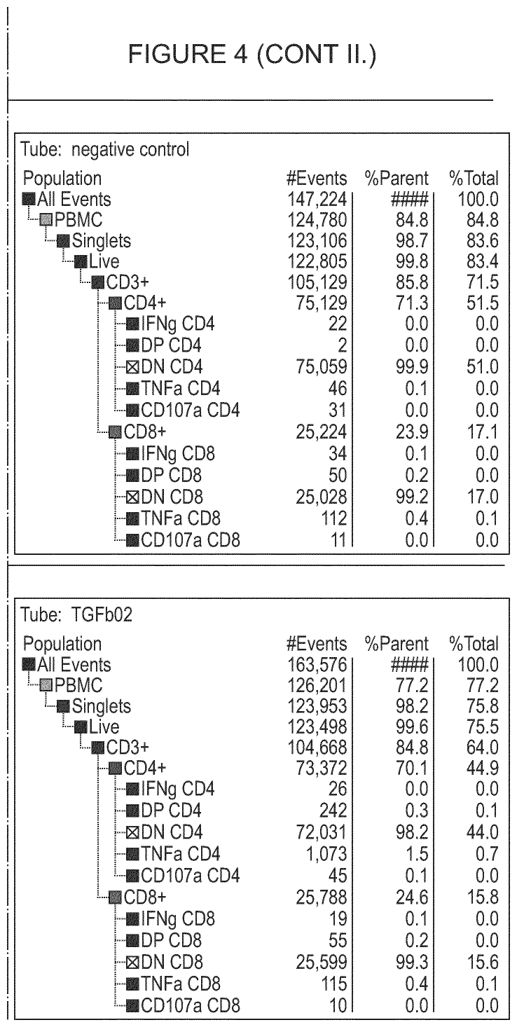

Intracellular Cytokine Staining (ICS) analysis was carried to further characterise the functionality of T cells responding to TGFb1 epitopes. In this example, the PBMCs from a healthy donor (BC-M-41) were thawed and stimulated with TGFb-02 (SEQ ID NO: 6), 13 days prior to the assay. IL-2 was added one day after the culture was set up (at 120 U/mL) and three days before the ICS was set up (at 60 U/mL). FACS analysis was performed and the live cell populations gated based on CD3⁺ CD4⁺ T cell fractions or CD3⁺ CD8⁺ T cell fractions. The expression of cytokine expression (IFNγ and TNFα) as well as marker for cytotoxicity (CD107a) were quantified. The FACS plots for the cytokine analysis are shown on the left-hand side of FIG. 4 while the percentages of the respective populations are summarised in the hierarchy table on the right of FIG. 4.

The CD3⁺ CD4⁺ T cell fraction (and not CD3⁺ CD8⁺ T cell fraction) was found to be reactive to TGFb-02 (SEQ ID NO: 6) as indicated by secretion of TNFα (either alone or in combination with IFNγ) with no/low expression of CD107a.

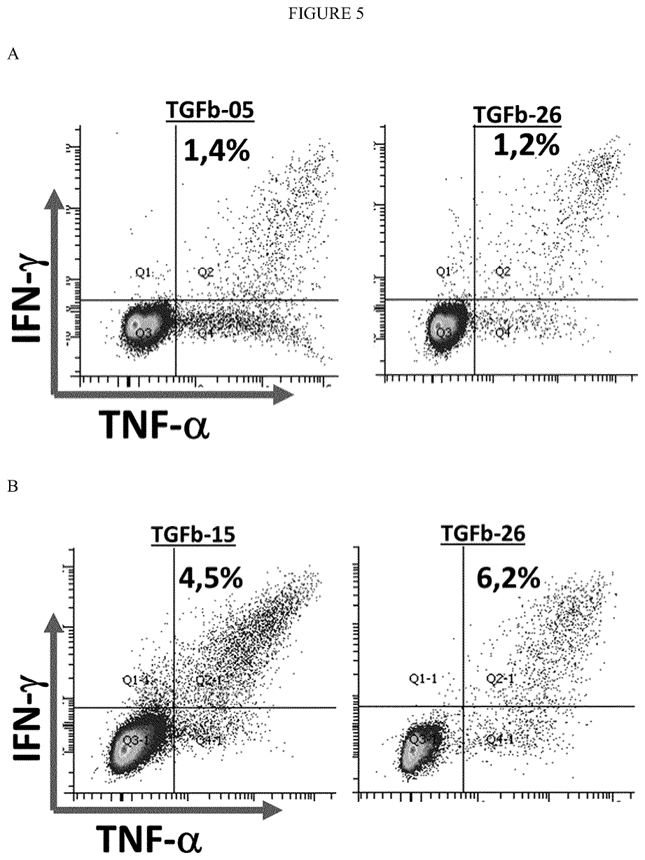

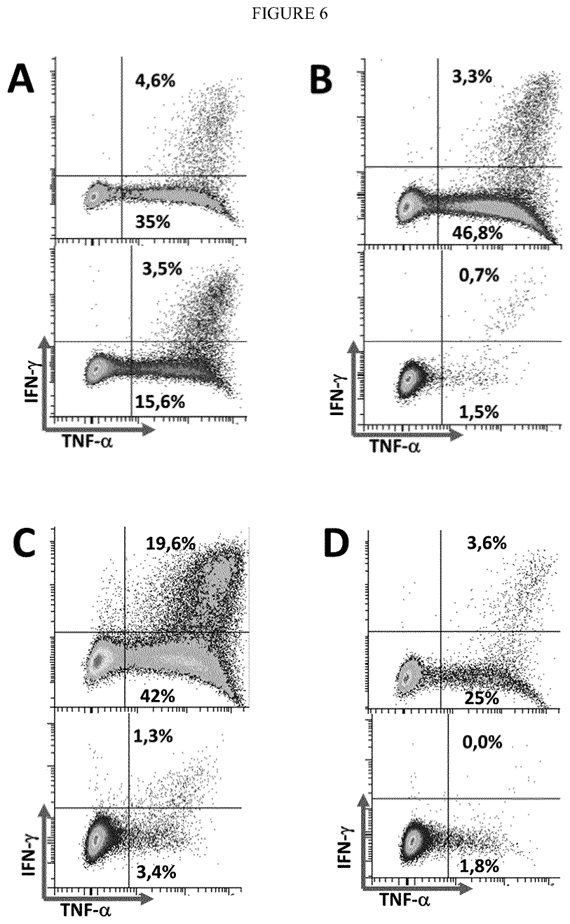

ICS was also used to show that epitopes TGFb-05 (SEQ ID NO: 12) and TGFb-26 (SEQ ID NO: 42) triggered both CD4⁺ (FIG. 5A) and CD8⁺ T-cell responses (FIG. 5B). Strong CD4⁺ and CD8⁺ T-cell responses were also detected against several of the lead epitopes after enrichment of specific cells by magnetically activated cell sorting (MACS) (FIG. 6), demonstrating the high immunogenic potential of several epitopes in TGFβ.

### Example 5—Identification of Ex Vivo Responses to TGFIβ Epitopes

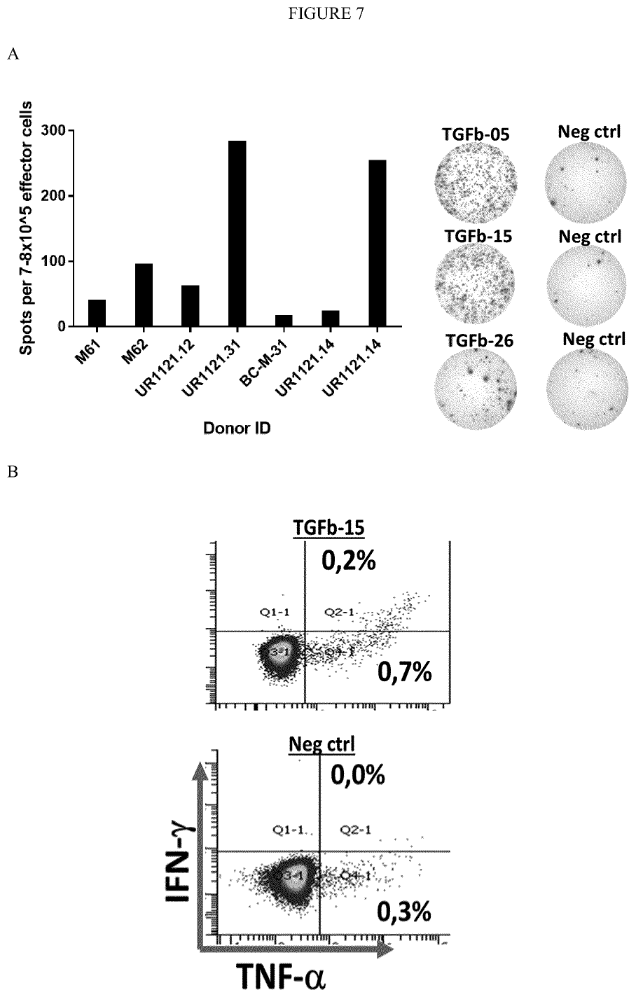

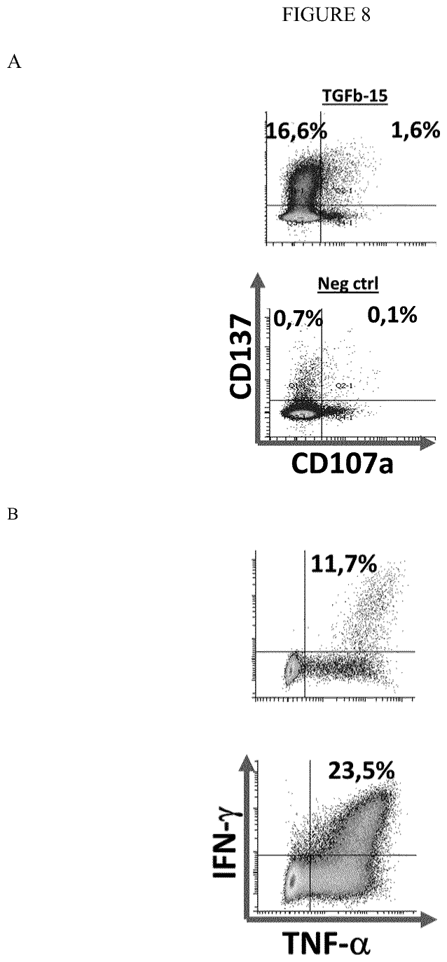

Ex vivo responses against several epitopes by PBMCs from both healthy subjects and cancer patient PBMCs. Cells were thawed and rested overnight before being plated and then stimulated for 48 hours. Both healthy and patient cells released significant amounts of IFN-γ (FIG. 7A), proving that cells from both healthy subjects and cancer patients harbored a high amount of freely circulating TGFβ-specific T cells. Most surprisingly, a CD8⁺ T-cell response was detected in ex vivo—plated PBMCs from a patient with prostate cancer after only 5 hours of stimulation with the epitope TGFb-15 (FIG. 7B). This finding suggested that this patient had a high fraction of circulating TGFβ-specific cytotoxic T cells. Given this strong response to TGFb-15, a specific T-cell culture against TGFb-15 was established using PBMCs from this patient. First, it was established that CD137 could be used as an activation marker for sorting specific T cells from this donor. Patient PBMCs were then stimulated with TGFb-15 and maintained the cells in culture for 14 days, after which PBMCs were re-stimulated with TGFb-15 for 18 hours and then analyzed for expression of CD137 and CD107a using fluorescent-activated cell sorting (FACS). This experiment demonstrated that CD137 is a suitable marker to enrich for specific T cells, given that 16.6% of CD8⁺ T cells were CD137⁺ after stimulation with the peptide (FIG. 8A).

TGFb-15-specific T cells were enriched using a MACS CD137 enrichment kit and were used to established a culture containing both CD4⁺ and CD8⁺ T cells specific for TGFb-15 (FIG. 8B). TGFβ-specific T-cell responses were assessed using this culture.

### Example 6—TGFb-15-Specific T Cells Can Recognize and Kill Cancer Cells

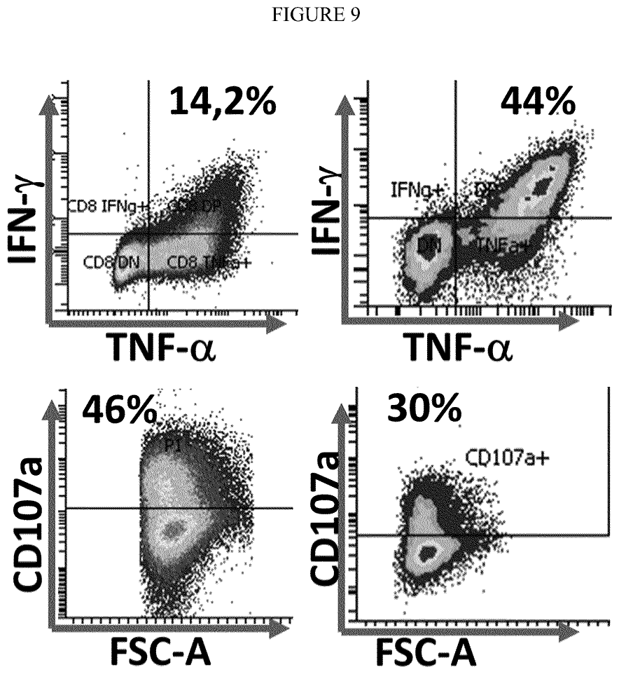

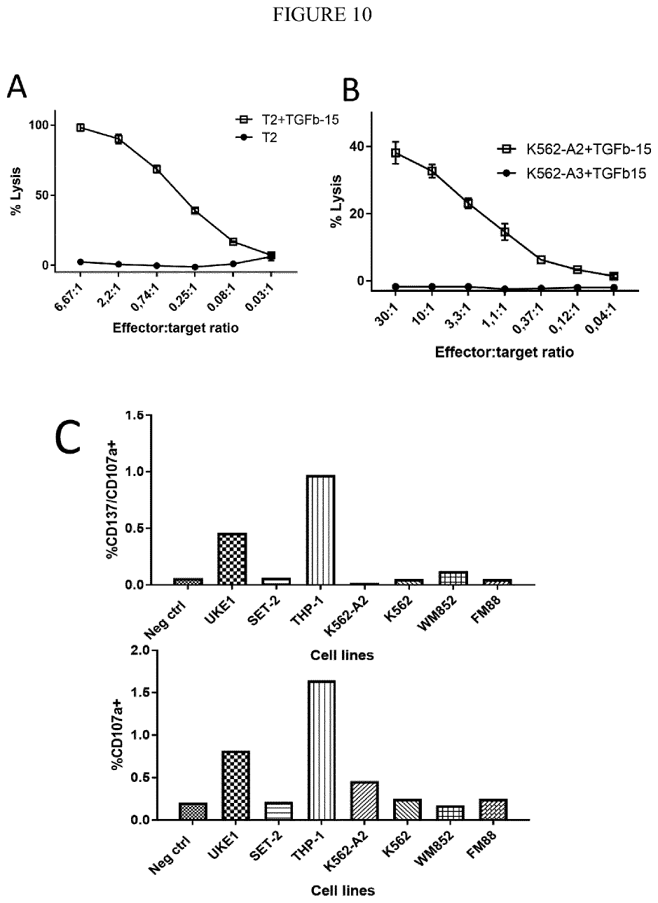

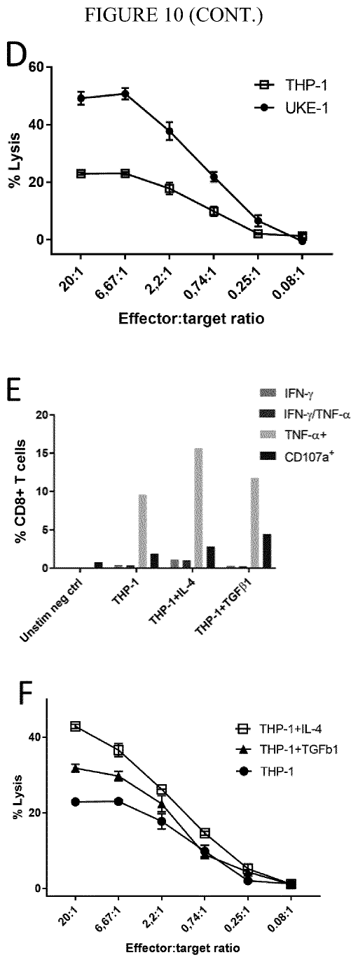

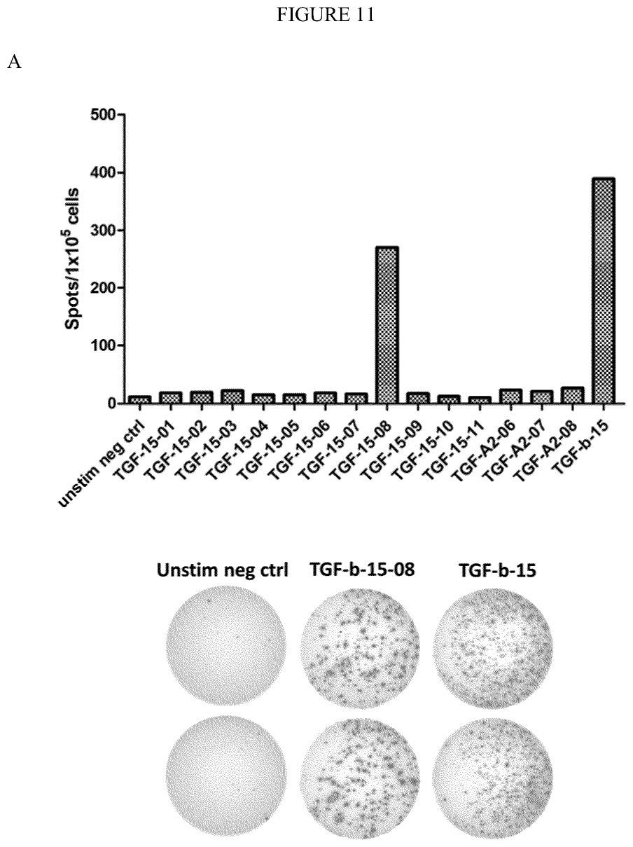

Using limiting dilution, CD8⁺ TGFb-15-specific T cells clones were established from the TGFβ described in Example 5. The CD8⁺ TGFb-15-specific clones showed high reactivity against TGFb-15 (FIG. 9). Staining of PBMCs from this patient with an HLA-A2⁺-specific antibody revealed that the donor was HLA-A2⁺ (data not shown). Next, standard chromium-51 cytotoxicity assays were performed to examine whether the specific T cells could lyse peptide-pulsed HLA-A2⁺ target cells. Peptide-pulsed HLA-A2⁺ T2 cells were readily lysed by the specific T cells, whereas un-pulsed T2 cells were not killed (FIG. 10A).

T2 cells are not only HLA-A2⁺, so the killing of these cells could potentially be mediated by a match on another HLA allele. For this reason, a further experiment was performed using K562 cells as targets. The original K562-line is HLA-deficient, but these experiments were performed with two lines genetically modified to stably express either HLA-A2 or HLA-A3. This ensured that these were the only HLA-alleles that the respective cells expressed. Only peptide-pulsed HLA-A2⁺ K562 cells were killed by the TGFb-15-specific clones, whereas un-pulsed HLA-A2⁺ K562 cells and peptide-pulsed HLA-A3⁺ K562 cells were not recognized (FIG. 10B).

Almost all cells can secrete TGFβ, which is heavily involved in creating a tumor-suppressive environment. For this reason, it was investigated whether the TGFb-15-specific T cells could recognize HLA-A2⁺ cancer cell lines. The cell lines UKE-1, SET-2, and THP-1, which are all derived from patients with acute myeloid leukemia (AML) were used as the target cells, along with two HLA-A2⁺ melanoma cell lines (WM852 and FM88) and K562 and HLA-A2⁺ K562 cells. TGFb-15-specific T cells were stimulated overnight with the respective target cells at an effector:target ratio of 3:1. The specific T cells recognized the two cancer cell lines THP-1 and UKE-1, whereas the other cell lines did not activate the T cells (FIG. 10C). Moreover, a chromium-51 cytotoxicity experiment revealed that TGFb-15-specific T cells killed both UKE-1 and THP-1 cells (FIG. 10D).

The THP-1 cell line is relatively undifferentiated line, and treatment with different cytokines can affect gene expression in these cells. Interleukin (IL)-4 is a cornerstone cytokine in development of the Th2-response. It was therefore speculated that treatment of THP-1 cells with IL-4 might increase TGFβ expression by these cells. Additionally, because TGFβ generates a positive feedback loop for its intracellular production, it was speculated that treatment of THP-1 cells with TGFβ also would induce TGFβ expression. Of note, the target epitope TGFb-15 is expressed in the LAP peptide part of the TGFβ pre-cursor protein and not in the mature active form of TGFβ (see FIG. 1E). Thus, pre-treatment of THP-1 cells with active TGFβ would not add the recognized epitope to the THP-1 cells but only increase intracellular production of TGFβ.

THP-1 cells treated with either IL-4 or TGFβ for 48 hours were used to stimulate TGFb-15-specific CD8⁺ T cells for 18 hours. It was demonstrated that cytokine-treated THP-1 cells induced greater activation of the TGFb-15-specific T cells compared to unstimulated THP-1 cells (FIG. 10E). Ultimately, it was shown that cytokine stimulation of THP-1 cells with either IL-4 or TGFβ enhanced the number of lysed cells (FIG. 10F).

### Example 7—Identification of a Minimal TGFβ Epitope Sequence

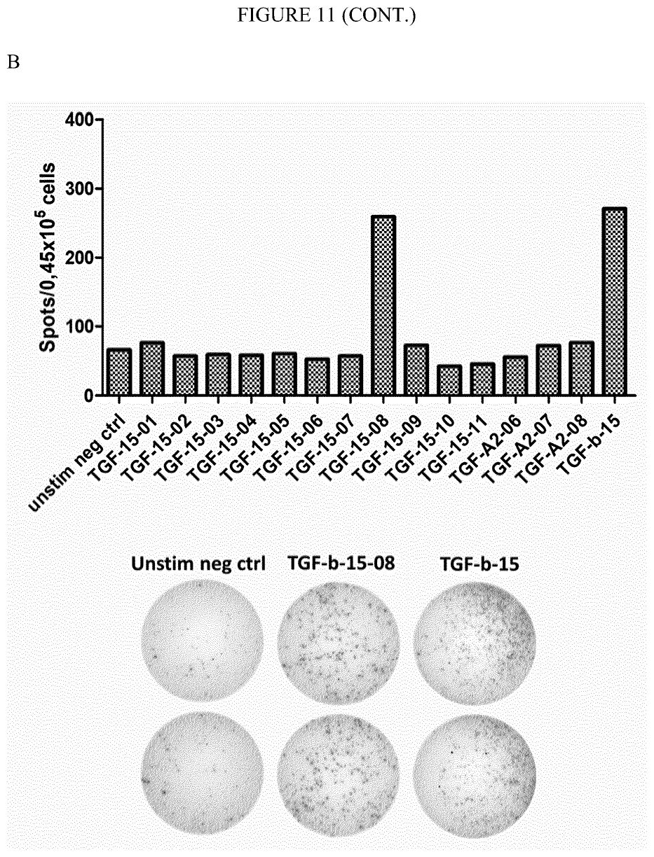

Since the TGFb-15 epitope is a 20-mer, it cannot be presented in its full length on HLA-I molecules. Accordingly, further experiments were carried to determine the minimal epitope sequence recognized by the TGFb-15-specific T cells. Specifically, the TGFb-15 epitope sequence was divided epitope into a 9 mer peptide library with eight overlapping amino acids, thus generating 12 9 mer peptides. T cells from the TGFb-15-specific bulk culture used to generate the TGFb-15-specific CD8⁺ T cell clones were plated in ELISPOT and stimulated with each of the 9 mer peptides. The results showed that the minimal epitope within the TGFb-15 sequence was the sequence VLLSRAELRL (TGFb-15short; SEQ ID NO: 66) (see FIG. 11).

### Example 8—a Decamer Epitope in the Signal Peptide of TGFβ is a Target of Specific T Cells

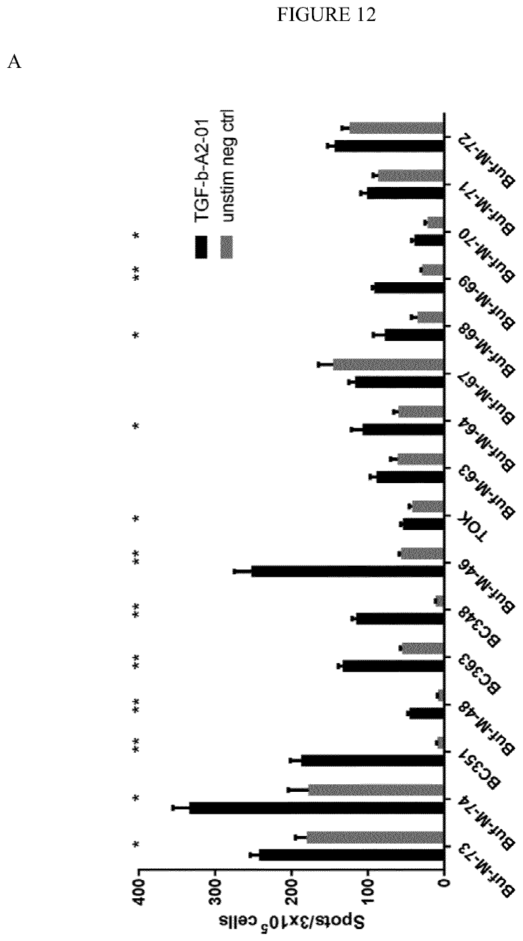

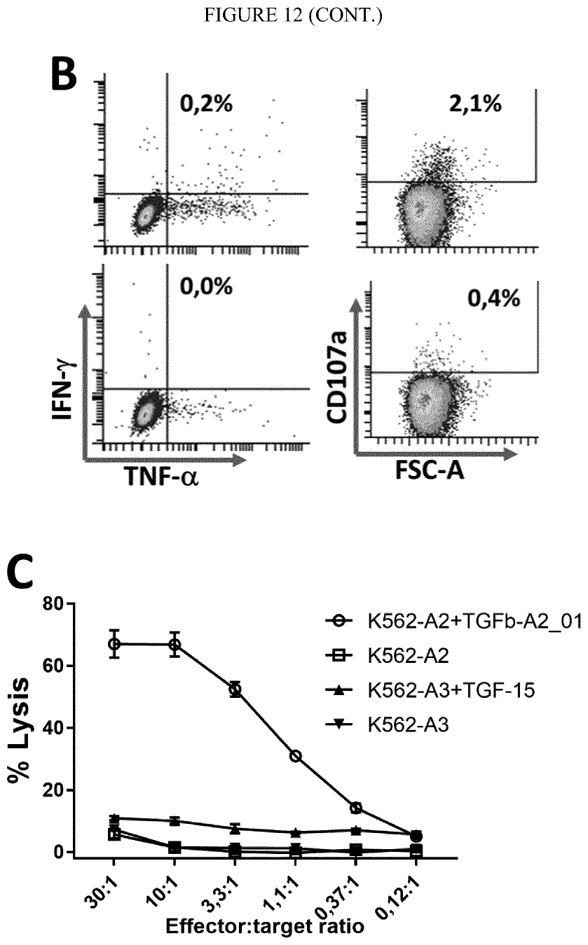

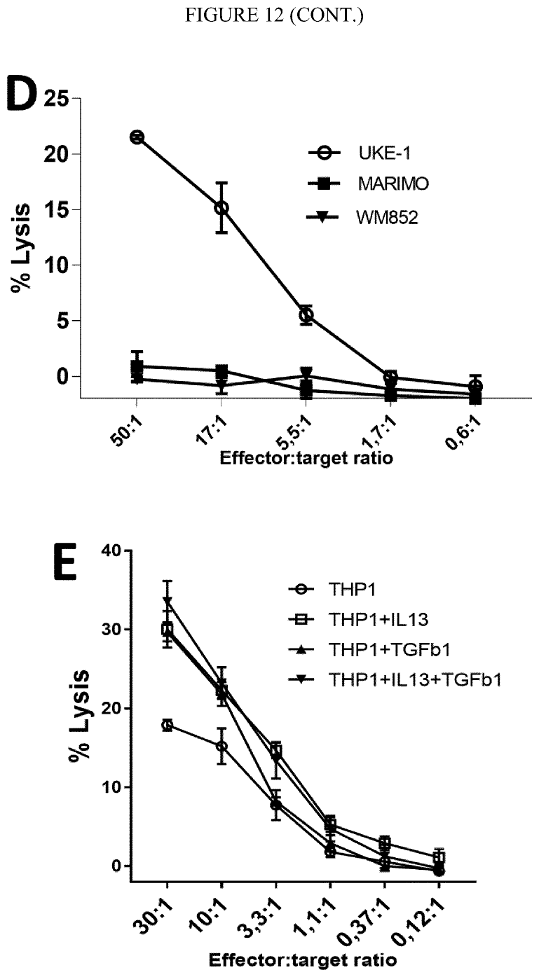

Given the high frequency of CD8⁺ T-cell responses against several 20-mer epitopes within the TGFβ sequence, the inventors sought to identify other HLA-A2-restricted decamer epitopes. Using the SYFPEITHI database of MHC ligands and peptide motifs Rammensee et al. (SYFPEITHI: database for MHC ligands and peptide motifs; www.syfpeithi.de.; accessed Oct. 30, 2014), the entire TGFβ sequence was searched for decamer epitopes with a high binding affinity to HLA-A2. The peptide sequence LLLLLPLLWL (TGFb-A2-01; SEQ ID NO: 67) emerged as the top binding decamer epitope, with a binding affinity score of 30. Spontaneous T-cell responses against the TGFb-A2-01 epitope by HLA-A2⁺PBMCs derived from healthy subjects were then investigated. Surprisingly, the majority of PBMCs displayed a response against the TGFb-A2-01 epitope (FIG. 12A). Using ICS, it was confirmed that these responses were from CD8⁺ T-cells (FIG. 12B).

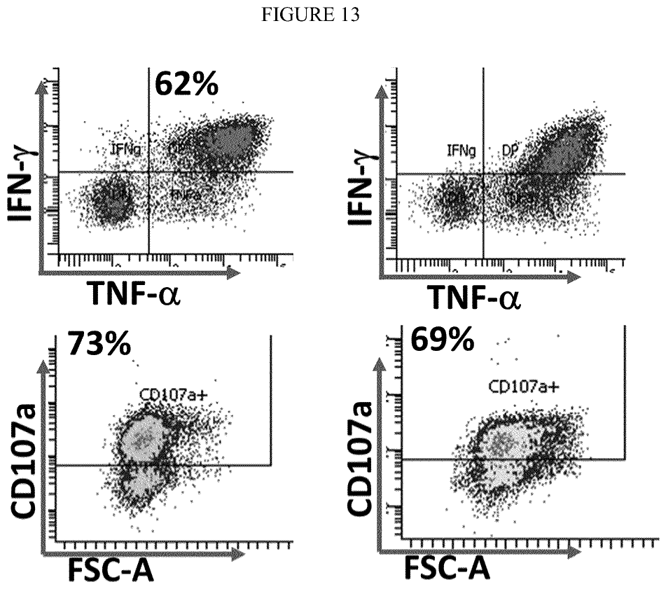

TGFb-A2-01-specific T cells were then isolated from a healthy subject (BC363) with a solid response to TGFb-A2-01 by performing one in vitro stimulation of PBMCs from said subject followed by 14 days of culture. Next, the PBMCs were stimulated overnight with TGFb-A2-01. Specific T cells were then enriched using FACS with gating on CD3⁺, CD8⁺, CD137⁺ cells. The enriched cells were expanded as described in Example 1. After 14 days of culture, several cell lines showed high specificity for the TGFb-A2-01 peptide (FIG. 13).

The ability of the TGFb-A2-01-specific T cells to lyse peptide-pulsed HLA-A2⁺ K562 target cells was tested in a standard Cr51 cytotoxicity assay. Peptide-pulsed HLA-A2⁺ K562 cells were lysed, whereas un-pulsed HLA-A2⁺ and peptide-pulsed HLA-A3⁺ target cells were not (FIG. 12C).

Since the TGFb-15-specific T cells described above killed the AML cell lines UKE-1 and THP-1, it was tested whether the TGFb-A2-01 specific clones could also kill these target cancer cells. UKE-1 and THP-1 cancer cells were readily killed by the TGFb-A2-01-specific T cells (see FIGS. 12D and E). Additionally, stimulation of THP-1 cells with IL-13, TGFβ, or both IL-13 and TGFβ in combination enhanced the fraction of killed target cells (FIG. 12E).

### Conclusions

TGFb1 is a crucial enforcer of immune homeostasis and tolerance, inhibiting the expansion and function of many components of the immune system. Perturbations in TGFb1 signalling underlie inflammatory diseases and promote tumour emergence. TGFb1 is also central to immune suppression within the tumour microenvironment, and recent studies have revealed roles in tumour immune evasion and poor responses to cancer immunotherapy. Expression of TGFb1 is a main characteristic of both tumour associated macrophages (TAMs) and myeloid-derived suppressor cells. TGFb1-expressing cells also play a major role in the development of an immune-inhibitory microenvironment because they prevent effector lymphocyte proliferation at the tumour site. Activation of TGFb1-specific T cells by vaccination, for example, should therefore cause T cell infiltration at the tumour site.

For the first time, it has been found that TGFb1-specific effector T cells could be exploited to specifically target TGFb1-expressing cells. In particular, the present inventors have identified peripheral TGFb1-specific T cells that were naturally present in both cancer patients and healthy donors by screening a peptide library covering the entire amino acid sequence of TGFb1. Interestingly, it has been discovered that TGFb1 contains multiple epitopes that were frequently recognised by peripheral T cells spread in different regions on the TGFb1 sequence.

Frequent T-cell responses against TGFb1 were observed, which underlines the surprising finding that TGFb1 is highly immunogenic. It is particularly unexpected that TGF1b would be highly immunogenic to the degree observed by the present inventors, given that TGFb1 is so central to immune suppression. Further to this, regions of TGFb1 capable of generating particularly strong immune responses have been identified and these would be ideal for use in a peptide-based vaccination approach.

The present inventors findings, which are surprising given the role of TGFb1 in the suppression of the immune system e.g. in the TME, suggests that it would be possible to boost a TGFb1-specific immune response in most patients with solid tumours as well as haematological malignancies.

Many different therapeutic strategies focus on targeting the immune suppressive tumour microenvironment (TME) with the aim to deplete or reprogram the immune suppressive cells or target the functional mediators secreted by these cells. The surprising results discussed above demonstrate that an immune modulatory vaccination targeting TGFb1 would be an effective way of targeting immune suppressive cells in the TME. In contrast to the other clinical strategies, this unique approach combines both depletion of immune suppressive cells, including cancer cells (through direct killing by cytotoxic T cells) and reprogramming of immune suppressive cell populations (by introducing pro-inflammatory cytokines into the immune suppressive microenvironment). TGFb1 specific T cells can specifically react to immune suppressive cells as TGFb1 expression is a major contributor to the phenotype of immune suppressive cells. TGFb1 vaccines that the rebalance the microenvironment should increase the effect of T cell-enhancing drugs, such as checkpoint blockers like anti-PD1 antibodies. Combination therapy with TGFb1 vaccines and checkpoint blocking antibodies should therefore increase the number of patients who could respond to therapy.

In conclusion, the experimental results discussed above provide a valuable approach for directly targeting the major contributor for the lack of immune responses in most patients with cancer: TGFb1.

SEQUENCES

```
Full-length human TGFb1 pre-protein (NP_000651.3)(SEQ ID NO: 1):
                10 20 30 40 50
MPPSGLRLLL LLLPLLWLLV LTPGRPAAGL STCKTIDMEL VKRKRIEAIR
                60 70 80 90 100
GQILSKLRLA SPPSQGEVPP GPLPEAVLAL YNSTRDRVAG ESAEPEPEPE
              110 120 130 140 150
ADYYAKEVTR VLMVETHNEI YDKFKQSTHS IYMFFNTSEL REAVPEPVLL
              160 170 180 190 200
SRAELRLLRL KLKVEQHVEL YQKYSNNSWR YLSNRLLAPS DSPEWLSFDV
              210 220 230 240 250
TGVVRQWLSR GGEIEGFRLS AHCSCDSRDN TLQVDINGFT TGRRGDLATI
              260 270 280 290 300
HGMNRPFLLL MATPLERAQH LQSSRHRRAL DTNYCFSSTE KNCCVRQLYI
              310 320 330 340 350
DFRKDLGWKW IHEPKGYHAN FCLGPCPYIW SLDTQYSKVL ALYNQHNPGA
              360 370 380 390
SAAPCCVPQA LEPLPIVYYV GRKPKVEQLS NMIVRSCKCS
```

In Table 1 below, “Start pos” and “End pos” indicate the positions within full length human TGFb1 pre-protein (SEQ ID NO: 1) unless otherwise indicated.

TABLE 1

| SEQ | | | Start | End |
|---|---|---|---|---|
| ID NO | Name | Sequence | pos | pos |
| 2 | TGFb1 signal | MPPSGLRLLLLLLPLLWLLVLTPGRPAAG | 1 | 29 |
| | peptide | | | |
| 3 | TGFb1 LAP | LSTCKTIDMELVKRKRIEAIRGQILSKLRLASPPSQGEVPP | 30 | 278 |
| | | GPLPEAVLALYNSTRDRVAGESAEPEPEPEADYYAKEVTRV | | |
| | | LMVETHNEIYDKFKQSTHSIYMFFNTSELREAVPEPVLLSR | | |
| | | AELRLLRLKLKVEQHVELYQKYSNNSWRYLSNRLLAPSDSP | | |
| | | EWLSFDVTGVVRQWLSRGGEIEGFRLSAHCSCDSRDNTLQV | | |
| | | DINGFTTGRRGDLATIHGMNRPFLLLMATPLERAQHLQSSR | | |
| | | HRR | | |
| 4 | Mature TGFb1 | ALDTNYCFSSTEKNCCVRQLYIDFRKDLGWKWIHEPKGYHA | 279 | 390 |
| | | NFCLGPCPYIWSLDTQYSKVLALYNQHNPGASAAPCCVPQA | | |
| | | LEPLPIVYYVGRKPKVEQLSNMIVRSCKCS | | |
| 5 | TGFb-01 | MPPSGLRLLLLLLPLLWLLV | 1 | 20 |
| 67 | TGFb-A2-01 | LLLLLPLLWL | 9 | 18 |
| 6 | TGFb-02 | LLLPLLWLLVLTPGRPAAGL | 11 | 30 |
| 7 | TGFb-02.1 | LLLPLLWLLVLTPGRPAAGLSTCKT | 11 | 35 |
| 8 | TGFb-02.2 | LRLLLLLLPLLWLLVLTPGRPAAGL | 6 | 30 |
| 9 | TGFb-02.3 | LRLLLLLLPLLWLLVLTPGRPAAGLSTCKT | 6 | 35 |
| 10 | TGFb-03 | LTPGRPAAGLSTCKTIDMEL | 21 | 40 |
| 11 | TGFb-04 | STCKTIDMELVKRKRIEAIR | 31 | 50 |
| 12 | TGFb-05 | VKRKRIEAIRGQILSKLRLA | 41 | 60 |
| 13 | TGFb-05.1 | IDMELVKRKRIEAIRGQILSKLRLA | 36 | 60 |
| 14 | TGFb-05.2 | VKRKRIEAIRGQILSKLRLASPPSQ | 41 | 65 |
| 15 | TGFb-05.3 | IDMELVKRKRIEAIRGQILSKLRLASPPSQ | 36 | 65 |
| 16 | TGFb-06 | GQILSKLRLASPPSQGEVPP | 51 | 70 |
| 17 | TGFb-07 | SPPSQGEVPPGPLPEAVLAL | 61 | 80 |
| 18 | TGFb-08 | GPLPEAVLALYNSTRDRVAG | 71 | 90 |
| 19 | TGFb-09 | YNSTRDRVAGESAEPEPEPE | 81 | 100 |
| 20 | TGFb-10 | ESAEPEPEPEADYYAKEVTR | 91 | 110 |
| 21 | TGFb-11 | ADYYAKEVTRVLMVETHNEI | 101 | 120 |
| 22 | TGFb-12 | VLMVETHNEIYDKFKQSTHS | 111 | 130 |
| 23 | TGFb-13 | YDKFKQSTHSIYMFFNTSEL | 121 | 140 |
| 24 | TGFb-13.1 | THNEIYDKFKQSTHSIYMFFNTSEL | 116 | 140 |
| 25 | TGFb-13.2 | YDKFKQSTHSIYMFFNTSELREAVP | 121 | 145 |
| 26 | TGFb-13.3 | THNEIYDKFKQSTHSIYMFFNTSELREAVP | 116 | 145 |
| 27 | TGFb-14 | IYMFFNTSELREAVPEPVLL | 131 | 150 |
| 28 | TGFb-15 | REAVPEPVLLSRAELRLLRL | 141 | 160 |
| 66 | TGFb-15short | VLLSRAELRL | 148 | 167 |
| 29 | TGFb-15.1 | NTSELREAVPEPVLLSRAELRLLRL | 136 | 160 |
| 30 | TGFb-15.2 | REAVPEPVLLSRAELRLLRLKLKVE | 141 | 165 |
| 31 | TGFb-15.3 | NTSELREAVPEPVLLSRAELRLLRLKLKVE | 136 | 165 |
| 32 | TGFb-16 | SRAELRLLRLKLKVEQHVEL | 151 | 170 |
| 33 | TGFb-17 | KLKVEQHVELYQKYSNNSWR | 161 | 180 |
| 34 | TGFb-18 | YQKYSNNSWRYLSNRLLAPS | 171 | 190 |
| 35 | TGFb-19 | YLSNRLLAPSDSPEWLSFDV | 181 | 200 |
| 36 | TGFb-20 | DSPEWLSFDVTGVVRQWLSR | 191 | 210 |
| 37 | TGFb-21 | TGVVRQWLSRGGEIEGFRLS | 201 | 220 |
| 38 | TGFb-22 | GGEIEGFRLSAHCSCDSRDN | 211 | 230 |
| 39 | TGFb-23 | AHCSCDSRDNTLQVDINGFT | 221 | 240 |
| 40 | TGFb-24 | TLQVDINGFTTGRRGDLATI | 231 | 250 |
| 41 | TGFb-25 | TGRRGDLATIHGMNRPFLLL | 241 | 260 |
| 42 | TGFb-26 | HGMNRPFLLLMATPLERAQH | 251 | 270 |
| 43 | TGFb-26.1 | DLATIHGMNRPFLLLMATPLERAQH | 246 | 270 |
| 44 | TGFb-26.2 | HGMNRPFLLLMATPLERAQHLQSSR | 251 | 275 |
| 45 | TGFb-26.3 | DLATIHGMNRPFLLLMATPLERAQHLQSSR | 246 | 275 |
| 46 | TGFb-27 | MATPLERAQHLQSSRHRRAL | 261 | 280 |
| 47 | TGFb-28 | LQSSRHRRALDTNYCFSSTE | 271 | 290 |
| 48 | TGFb-29 | DTNYCFSSTEKNCCVRQLYI | 281 | 300 |
| 49 | TGFb-30 | KNCCVRQLYIDFRKDLGWKW | 291 | 310 |
| 50 | TGFb-30.1 | FSSTEKNCCVRQLYIDFRKDLGWKW | 286 | 310 |
| 51 | TGFb-30.2 | KNCCVRQLYIDFRKDLGWKWIHEPK | 291 | 315 |
| 52 | TGFb-30.3 | FSSTEKNCCVRQLYIDFRKDLGWKWIHEPK | 286 | 315 |
| 53 | TGFb-31 | DFRKDLGWKWIHEPKGYHAN | 301 | 320 |
| 54 | TGFb-32 | IHEPKGYHANFCLGPCPYIW | 311 | 330 |
| 55 | TGFb-33 | FCLGPCPYIWSLDTQYSKVL | 321 | 340 |
| 56 | TGFb-33.1 | GYHANFCLGPCPYIWSLDTQYSKVL | 316 | 340 |
| 57 | TGFb-33.2 | FCLGPCPYIWSLDTQYSKVLALYNQ | 321 | 345 |
| 58 | TGFb-33.3 | GYHANFCLGPCPYIWSLDTQYSKVLALYNQ | 316 | 345 |
| 59 | TGFb-34 | SLDTQYSKVLALYNQHNPGA | 331 | 350 |
| 60 | TGFb-35 | ALYNQHNPGASAAPCCVPQA | 341 | 360 |
| 61 | TGFb-36 | SAAPCCVPQALEPLPIVYYV | 351 | 370 |
| 62 | TGFb-37 | LEPLPIVYYVGRKPKVEQLS | 361 | 380 |
| 63 | TGFb-38 | GRKPKVEQLSNMIVRSCKCS | 371 | 390 |
| 64 | TGFb-38.1 | IVYYVGRKPKVEQLSNMIVRSCKCS | 365 | 390 |
| 65 | TGFb1 LAP | YDKFKQSTHSIYMFFNTSELREAVPEPVLLSRAELRLLRL | 121 | 160 |
| | sub-region | | | |

1. A polypeptide which is an immunogenic fragment of human transforming growth factor 1 (TGFb1) and which comprises or consists of a sequence of at least 9 consecutive amino acids of SEQ ID NO: 1.

2. The polypeptide of claim 1, which comprises or consists of up to 9, 10, 11, 12, 13, 14, 15, 16, 17, 18, 19, 20, 21, 22, 23, 24, 25, 26, 27, 28, 29, 30, 35, 40, 45 or 50 consecutive amino acids of SEQ ID NO: 1.

3. The polypeptide of claim 1 or 2, which comprises or consists of the amino acid sequence of any one of SEQ ID NOs: 66, 28-31, 67, 5-9, 42-45, 12-15, 55-58, 23-26, 49-52, 63, 64, 65 or 2.

4. The polypeptide of claim 3, which comprises or consists of the amino acid sequence of any one of SEQ ID NOs: 66, 28, 67, 5, 6, 42, 12, 55, 23, 49, or 63.

5. The polypeptide of any one of claims 1-4, which has a maximum length of 9, 10, 11, 12, 13, 14, 15, 16, 17, 18, 19, 20, 21, 22, 23, 24, 25, 26, 27, 28, 29, 30, 35, 40, 45 or 50 amino acids and/or in which the C terminal amino acid is replaced with the corresponding amide.

6. The polypeptide of any one of claims 1-5, which comprises a HLA-A2 restricted epitope, optionally wherein the HLA-A2-restricted epitope comprises or consists of the amino acid sequence of SEQ ID NO: 66 or 67.

7. A polynucleotide encoding a polypeptide according to any one of claims 1-6, optionally comprised within a vector.

8. A composition comprising a polypeptide according to any one of claims 1-6 and/or a polynucleotide according to claim 7; and optionally an adjuvant.

9. The composition of claim 8 further comprising at least one different polypeptide according to any one of claims 1-6; at least one different polynucleotide according to claim 7;
and/or at least one pharmaceutically acceptable diluent, carrier or preservative.

10. The composition of claim 8 or 9, which comprises an adjuvant selected from the group consisting of bacterial DNA based adjuvants, oil/surfactant based adjuvants, viral dsRNA based adjuvants, imidazochinilines, and a Montanide ISA adjuvant.

11. A method of treating or preventing a disease or condition in a subject, the method comprising administering to the subject the polypeptide of any one of claims 1-6, the polynucleotide of claim 7, and/or the composition of any one of claims 8-10.

12. The method of claim 11, wherein the disease or condition is:
(i) a cancer optionally selected from the group consisting of a breast cancer, a cervical cancer, a gastric cancer, a liver cancer, an ovarian cancer, a pancreatic cancer, a lung cancer (such as a non-small-cell lung carcinoma (NSCLC)), a melanoma, a leukemia (such as an acute myeloid leukemia), or a prostate cancer; and/or
(ii) characterized at least in part by inappropriate or excessive immune suppressive function of TGFb1-expressing cells and/or inappropriate or excessive expression of IL-4 and/or IL-13.

13. The method of claim 11 or 12, wherein the disease or condition is cancer and the method further comprises the simultaneous or sequential administration of an additional cancer therapy, preferably an antibody.

14. A method of stimulating TGFb1-specific T cells, the method comprising contacting the T cells with: the polypeptide of any one of claims 1-6; and/or a composition of any one of claims 8-10, wherein the composition comprises at least one polypeptide as defined in any one of claims 1-6.

15. The method of claim 14, wherein the T cells are present in a sample taken from a healthy subject or from a cancer patient, optionally a tumour sample.


SEQUENCE LISTING (67 sequences)

SEQ ID NO: 1 - protein, 390 aa, Homo sapiens
  PEPTIDE (1)..(390): Full-length human TGFb1 pre-protein (NP_000651.3)
MPPSGLRLLLLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 2 - protein, 29 aa, Homo sapiens
  PEPTIDE (1)..(29): TGFb1 signal peptide
MPPSGLRLLLLLLPLLWLLVLTPGRPAAG

SEQ ID NO: 3 - protein, 249 aa, Homo sapiens
  PEPTIDE (1)..(249): TGFb1 LAP
LSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRR

SEQ ID NO: 4 - protein, 112 aa, Homo sapiens
  PEPTIDE (1)..(112): Mature TGFb1
ALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 5 - protein, 20 aa, Homo sapiens
  PEPTIDE (1)..(20): TGFb-01
MPPSGLRLLLLLLPLLWLLV

SEQ ID NO: 6 - protein, 20 aa, Homo sapiens
  PEPTIDE (1)..(20): TGFb-02
LLLPLLWLLVLTPGRPAAGL

SEQ ID NO: 7 - protein, 25 aa, Homo sapiens
  PEPTIDE (1)..(25): TGFb-02.1
LLLPLLWLLVLTPGRPAAGLSTCKT

SEQ ID NO: 8 - protein, 25 aa, Homo sapiens
  PEPTIDE (1)..(25): TGFb-02.2
LRLLLLLLPLLWLLVLTPGRPAAGL

SEQ ID NO: 9 - protein, 30 aa, Homo sapiens
  PEPTIDE (1)..(30): TGFb-02.3
LRLLLLLLPLLWLLVLTPGRPAAGLSTCKT

SEQ ID NO: 10 - protein, 20 aa, Homo sapiens
  PEPTIDE (1)..(20): TGFb-03
LTPGRPAAGLSTCKTIDMEL

SEQ ID NO: 11 - protein, 20 aa, Homo sapiens
  PEPTIDE (1)..(20): TGFb-04
STCKTIDMELVKRKRIEAIR

SEQ ID NO: 12 - protein, 20 aa, Homo sapiens
  PEPTIDE (1)..(20): TGFb-05
VKRKRIEAIRGQILSKLRLA

SEQ ID NO: 13 - protein, 25 aa, Homo sapiens
  PEPTIDE (1)..(25): TGFb-05.1
IDMELVKRKRIEAIRGQILSKLRLA

SEQ ID NO: 14 - protein, 25 aa, Homo sapiens
  PEPTIDE (1)..(25): TGFb-05.2
VKRKRIEAIRGQILSKLRLASPPSQ

SEQ ID NO: 15 - protein, 30 aa, Homo sapiens
  PEPTIDE (1)..(30): TGFb-05.3
IDMELVKRKRIEAIRGQILSKLRLASPPSQ

SEQ ID NO: 16 - protein, 20 aa, Homo sapiens
  PEPTIDE (1)..(20): TGFb-06
GQILSKLRLASPPSQGEVPP

SEQ ID NO: 17 - protein, 20 aa, Homo sapiens
  PEPTIDE (1)..(20): TGFb-07
SPPSQGEVPPGPLPEAVLAL

SEQ ID NO: 18 - protein, 20 aa, Homo sapiens
  PEPTIDE (1)..(20): TGFb-08
GPLPEAVLALYNSTRDRVAG

SEQ ID NO: 19 - protein, 20 aa, Homo sapiens
  PEPTIDE (1)..(20): TGFb-09
YNSTRDRVAGESAEPEPEPE

SEQ ID NO: 20 - protein, 20 aa, Homo sapiens
  PEPTIDE (1)..(20): TGFb-10
ESAEPEPEPEADYYAKEVTR

SEQ ID NO: 21 - protein, 20 aa, Homo sapiens
  PEPTIDE (1)..(20): TGFb-11
ADYYAKEVTRVLMVETHNEI

SEQ ID NO: 22 - protein, 20 aa, Homo sapiens
  PEPTIDE (1)..(20): TGFb-12
VLMVETHNEIYDKFKQSTHS

SEQ ID NO: 23 - protein, 20 aa, Homo sapiens
  PEPTIDE (1)..(20): TGFb-13
YDKFKQSTHSIYMFFNTSEL

SEQ ID NO: 24 - protein, 25 aa, Homo sapiens
  PEPTIDE (1)..(25): TGFb-13.1
THNEIYDKFKQSTHSIYMFFNTSEL

SEQ ID NO: 25 - protein, 25 aa, Homo sapiens
  PEPTIDE (1)..(25): TGFb-13.2
YDKFKQSTHSIYMFFNTSELREAVP

SEQ ID NO: 26 - protein, 30 aa, Homo sapiens
  PEPTIDE (1)..(30): TGFb-13.3
THNEIYDKFKQSTHSIYMFFNTSELREAVP

SEQ ID NO: 27 - protein, 20 aa, Homo sapiens
  PEPTIDE (1)..(20): TGFb-14
IYMFFNTSELREAVPEPVLL

SEQ ID NO: 28 - protein, 20 aa, Homo sapiens
  PEPTIDE (1)..(20): TGFb-15
REAVPEPVLLSRAELRLLRL

SEQ ID NO: 29 - protein, 25 aa, Homo sapiens
  PEPTIDE (1)..(25): TGFb-15.1
NTSELREAVPEPVLLSRAELRLLRL

SEQ ID NO: 30 - protein, 25 aa, Homo sapiens
  PEPTIDE (1)..(25): TGFb-15.2
REAVPEPVLLSRAELRLLRLKLKVE

SEQ ID NO: 31 - protein, 30 aa, Homo sapiens
  PEPTIDE (1)..(30): TGFb-15.3
NTSELREAVPEPVLLSRAELRLLRLKLKVE

SEQ ID NO: 32 - protein, 20 aa, Homo sapiens
  PEPTIDE (1)..(20): TGFb-16
SRAELRLLRLKLKVEQHVEL

SEQ ID NO: 33 - protein, 20 aa, Homo sapiens
  PEPTIDE (1)..(20): TGFb-17
KLKVEQHVELYQKYSNNSWR

SEQ ID NO: 34 - protein, 20 aa, Homo sapiens
  PEPTIDE (1)..(20): TGFb-18
YQKYSNNSWRYLSNRLLAPS

SEQ ID NO: 35 - protein, 20 aa, Homo sapiens
  PEPTIDE (1)..(20): TGFb-19
YLSNRLLAPSDSPEWLSFDV

SEQ ID NO: 36 - protein, 20 aa, Homo sapiens
  PEPTIDE (1)..(20): TGFb-20
DSPEWLSFDVTGVVRQWLSR

SEQ ID NO: 37 - protein, 20 aa, Homo sapiens
  PEPTIDE (1)..(20): TGFb-21
TGVVRQWLSRGGEIEGFRLS

SEQ ID NO: 38 - protein, 20 aa, Homo sapiens
  PEPTIDE (1)..(20): TGFb-22
GGEIEGFRLSAHCSCDSRDN

SEQ ID NO: 39 - protein, 20 aa, Homo sapiens
  PEPTIDE (1)..(20): TGFb-23
AHCSCDSRDNTLQVDINGFT

SEQ ID NO: 40 - protein, 20 aa, Homo sapiens
  PEPTIDE (1)..(20): TGFb-24
TLQVDINGFTTGRRGDLATI

SEQ ID NO: 41 - protein, 20 aa, Homo sapiens
  PEPTIDE (1)..(20): TGFb-25
TGRRGDLATIHGMNRPFLLL

SEQ ID NO: 42 - protein, 20 aa, Homo sapiens
  PEPTIDE (1)..(20): TGFb-26
HGMNRPFLLLMATPLERAQH

SEQ ID NO: 43 - protein, 25 aa, Homo sapiens
  PEPTIDE (1)..(25): TGFb-26.1
DLATIHGMNRPFLLLMATPLERAQH

SEQ ID NO: 44 - protein, 25 aa, Homo sapiens
  PEPTIDE (1)..(25): TGFb-26.2
HGMNRPFLLLMATPLERAQHLQSSR

SEQ ID NO: 45 - protein, 30 aa, Homo sapiens
  PEPTIDE (1)..(30): TGFb-26.3
DLATIHGMNRPFLLLMATPLERAQHLQSSR

SEQ ID NO: 46 - protein, 20 aa, Homo sapiens
  PEPTIDE (1)..(20): TGFb-27
MATPLERAQHLQSSRHRRAL

SEQ ID NO: 47 - protein, 20 aa, Homo sapiens
  PEPTIDE (1)..(20): TGFb-28
LQSSRHRRALDTNYCFSSTE

SEQ ID NO: 48 - protein, 20 aa, Homo sapiens
  PEPTIDE (1)..(20): TGFb-29
DTNYCFSSTEKNCCVRQLYI

SEQ ID NO: 49 - protein, 20 aa, Homo sapiens
  PEPTIDE (1)..(20): TGFb-30
KNCCVRQLYIDFRKDLGWKW

SEQ ID NO: 50 - protein, 25 aa, Homo sapiens
  PEPTIDE (1)..(25): TGFb-30.1
FSSTEKNCCVRQLYIDFRKDLGWKW

SEQ ID NO: 51 - protein, 25 aa, Homo sapiens
  PEPTIDE (1)..(25): TGFb-30.2
KNCCVRQLYIDFRKDLGWKWIHEPK

SEQ ID NO: 52 - protein, 30 aa, Homo sapiens
  PEPTIDE (1)..(30): TGFb-30.3
FSSTEKNCCVRQLYIDFRKDLGWKWIHEPK

SEQ ID NO: 53 - protein, 20 aa, Homo sapiens
  PEPTIDE (1)..(20): TGFb-31
DFRKDLGWKWIHEPKGYHAN

SEQ ID NO: 54 - protein, 20 aa, Homo sapiens
  PEPTIDE (1)..(20): TGFb-32
IHEPKGYHANFCLGPCPYIW

SEQ ID NO: 55 - protein, 20 aa, Homo sapiens
  PEPTIDE (1)..(20): TGFb-33
FCLGPCPYIWSLDTQYSKVL

SEQ ID NO: 56 - protein, 25 aa, Homo sapiens
  PEPTIDE (1)..(25): TGFb-33.1
GYHANFCLGPCPYIWSLDTQYSKVL

SEQ ID NO: 57 - protein, 25 aa, Homo sapiens
  PEPTIDE (1)..(25): TGFb-33.2
FCLGPCPYIWSLDTQYSKVLALYNQ

SEQ ID NO: 58 - protein, 30 aa, Homo sapiens
  PEPTIDE (1)..(30): TGFb-33.3
GYHANFCLGPCPYIWSLDTQYSKVLALYNQ

SEQ ID NO: 59 - protein, 20 aa, Homo sapiens
  PEPTIDE (1)..(20): TGFb-34
SLDTQYSKVLALYNQHNPGA

SEQ ID NO: 60 - protein, 20 aa, Homo sapiens
  PEPTIDE (1)..(20): TGFb-35
ALYNQHNPGASAAPCCVPQA

SEQ ID NO: 61 - protein, 20 aa, Homo sapiens
  PEPTIDE (1)..(20): TGFb-36
SAAPCCVPQALEPLPIVYYV

SEQ ID NO: 62 - protein, 20 aa, Homo sapiens
  PEPTIDE (1)..(20): TGFb-37
LEPLPIVYYVGRKPKVEQLS

SEQ ID NO: 63 - protein, 20 aa, Homo sapiens
  PEPTIDE (1)..(20): TGFb-38
GRKPKVEQLSNMIVRSCKCS

SEQ ID NO: 64 - protein, 25 aa, Homo sapiens
  PEPTIDE (1)..(25): TGFb-38.1
IVYYVGRKPKVEQLSNMIVRSCKCS

SEQ ID NO: 65 - protein, 40 aa, Homo sapiens
  PEPTIDE (1)..(40): TGFb1 LAP sub-region
YDKFKQSTHSIYMFFNTSELREAVPEPVLLSRAELRLLRL

SEQ ID NO: 66 - protein, 10 aa, Homo sapiens
  PEPTIDE (1)..(10): TGFb-15short
VLLSRAELRL

SEQ ID NO: 67 - protein, 10 aa, Homo sapiens
  PEPTIDE (1)..(10): TGFb-A2-01
LLLLLPLLWL